(19) United States
(12) Patent Application Publication
(10) Pub. No.: US 2025/0090443 A1
(43) Pub. Date: Mar. 20, 2025

(54) SKIN CARE COMPOSITIONS AND THEIR USES

(71) Applicant: Flawless Beauty LLC, Ocean, NJ (US)

(72) Inventors: Jack GINDI, West Long Branch, NJ (US); Alleli PATULOT, Quezon City (PH); Ronalie GOPEZ, Quezon City (PH); Beverly BAUTISTA, London (GB)

(21) Appl. No.: 18/622,239

(22) Filed: Mar. 29, 2024

Related U.S. Application Data
(60) Provisional application No. 63/455,661, filed on Mar. 30, 2023.

(51) Int. Cl. A61K 8/67; A61K 8/34; A61K 8/49; A61K 8/55; A61K 8/64; A61K 8/73; A61K 8/9789; A61K 8/99; A61Q 19/02; A61Q 19/08

(52) U.S. Cl. CPC A61K 8/675; A61K 8/345; A61K 8/498; A61K 8/553; A61K 8/64; A61K 8/673; A61K 8/731; A61K 8/9789; A61K 8/99; A61Q 19/02; A61Q 19/08; A61K 2800/874

(57) ABSTRACT

The present invention provides a formulation comprising a component A and a component B, wherein the component A comprises: niacinamide; sodium hyaluronate; 1,2-Hexanediol; Sh-Oligopeptide-1; Sh-Oligopeptide-2; Sh-Polypeptide-1; Sh-Polypeptide-9; Sh-Polypeptide-11; dipeptide diaminobutyroyl benzylamide diacetate; and bacillus, folic acid, and/or soybean ferment extract; and wherein the component B comprises: niacinamide; tranexamic acid; rumex occidentalis extract; ascorbic acid; aminopropyl ascorbyl phosphate; sxifraga sarmentosa extract; arbutin; paeonia suffruticosa extract; scutellaria baicalensis root extract; and citric acid.

### CROSS-REFERENCE TO RELATED APPLICATION

This application claims priority of U.S. Provisional Application No. 63/455,661, filed Mar. 30, 2023, the contents of which are hereby incorporated by reference.

### FIELD OF INVENTION

This invention relates to the use of different formulations to treat skin conditions.

### BACKGROUND OF THE INVENTION

Hyperpigmentation, melasma, fine lines, and skin firmness are prevalent concerns that affect individuals of diverse ages and skin types. These dermatological conditions can be attributed to an array of factors, such as genetics, sun exposure, hormonal fluctuations, and aging.

The use of a firming composition and an anti-aging composition serum of this invention helps provide a solution for these issues.

The firming composition serum is a powerful blend of active ingredients that work together to enhance skin firmness and reduce signs of aging. Formulated with Niacinamide and HyaMoist® Care mini, this serum improves the skin's natural barrier function and provides deep hydration, resulting in a plump and supple appearance. Additionally, Glutathione, BioPlacenta, and SYN®-AKE promote cell regeneration and firm up the skin, resulting in a smoother and more youthful complexion with improved skin firmness.

The anti-aging composition serum targets dark spots, hyperpigmentation, and other skin discoloration issues using Clair Blanche II™, Niacinamide, Tyrostat™ 11, and Luzinci White. Clair Blanche II and Niacinamide reduce the appearance of dark spots and improve overall skin health. Tyrostat™ 11 and Luzinci White work together to inhibit melanin production, resulting in a brighter and more even complexion. With its effective ingredients, the anti-aging composition serum provides a comprehensive solution for correcting dark spots and revealing a more youthful and radiant appearance.

Further, dull, rough skin, and skin discoloration are common concerns among individuals with normal and combination skin.

The application of a refining composition and a brightening composition of this invention helps provide a solution to improve these skin concerns.

The refining composition serum's active ingredients, sh-oligopeptide-1, Niacinamide, Juventide®, and Vivensea™, improve skin texture and minimize fine lines by stimulating collagen production and enhancing the skin's natural barrier function, resulting in a more youthful and radiant appearance. Formulated with Alpha Arbutin, Vitamin C, Kojic Acid, and Glycolic Acid, the brightening composition's potent blend of active ingredients is carefully chosen for their skin-brightening and anti-aging properties, targeting dark spots, hyperpigmentation, and fine lines to improve the skin's overall appearance, revealing a brighter and more youthful complexion.

Moreover, acne scarring, and inflammatory marks are common concerns among individuals with oily and combination skin.

The application of an anti-scarring composition and the brightening composition of this invention helps provide a solution to improve these skin concerns.

The anti-scarring composition serum contains active ingredients such as Copper Tripeptide-1, sh-oligopeptide-1, and Snail Secretion Filtrate. These ingredients are renowned for their ability to improve the appearance of scars and the texture of the skin. Copper Tripeptide-1 stimulates collagen production, sh-oligopeptide-1 aids in skin repair, and Snail Secretion Filtrate contains Mucin and Glycosaminoglycan that can help regenerate skin cells, prevent damage, and has an excellent moisturizing ability.

The brightening composition serum contains Alpha Arbutin, Vitamin C, Kojic Acid, and Glycolic Acid as its ingredients. These ingredients provide skin-brightening and anti-aging properties, and can effectively reduce dark spots, hyperpigmentation, and fine lines. Alpha Arbutin and Kojic Acid have skin-lightening properties, while Vitamin C is an antioxidant that can protect the skin from environmental damage. Glycolic Acid, on the other hand, can help exfoliate dead skin cells and promote cell turnover.

### SUMMARY OF INVENTION

The disadvantages heretofore associated with the prior art are overcome by the present embodiments of skin care compositions and their uses as set forth in the claims.

The current invention provides a refining composition comprising a suitable amount of each of the following:
a) sh-Oligopeptide-1,
b) water,
c) glycerin,
d) niacinamide,
e) propylene glycol,
f) laminaria saccharina extract,
g) starfish coelomic fluid extract,
h) hydroxyethylcellulose (HEC),
i) disodium ethylenediaminetetraacetic acid (EDTA),
j) phenoxyethanol, and
k) ethylhexylglycerin.

The current invention provides a brightening composition comprising a suitable amount of each of the following:
a) water,
b) glycerin,
c) propylene glycol,
d) kojic acid,
e) glycolic acid,
f) potassium azelaoyl diglycinate,
g) sodium salt of pyroglutamic acid (sodium PCA),
h) arctostaphylos uva-ursi leaf extract,
i) magnesium ascorbyl phosphate,
j) alpha-arbutin,
k) hydroxyethylcellulose (HEC),
l) sodium benzoate,
m) sodium metabisulfite,
n) disodium ethylenediaminetetraacetic acid (EDTA),
o) phenoxyethanol, and
p) ethylhexylglycerin.

The present invention provides an anti-scarring composition comprising a suitable amount of each of the following:
a) sh-oligopeptide-1,
b) water,
c) snail secretion filtrate,
d) propylene glycol,
e) glycerin,
f) hydroxyethylcellulose (HEC),
g) copper tripeptide-1,
h) phenoxyethanol, and
i) ethylhexylglycerin.

The present invention provides a firming composition comprising a suitable amount of each of the following:
a) water,
b) Glycerin,
c) Niacinamide,
d) Butylene Glycol,
e) 1,2-Hexanediol,
f) Caprylyl Glycol,
g) Lecithin,
h) Dipeptide Diaminobutyroyl Benzylamide Diacetate,
i) Acetyl Glutamine,
j) Sh-Oligopeptide-1,
k) Sh-Oligopeptide-2,
l) Sh-Polypeptide-1,
m) Sh-Polypeptide-9,
n) Sh-Polypeptide-11,
o) Bacillus/Folic Acid/Soybean Ferment Extract,
p) Sodium Hyaluronate,
q) Glutathione,
r) Hydroxyethylcellulose,
s) Disodium EDTA,
t) Phenoxyethanol, and
u) Ethylhexylglycerin.

The present invention provides an anti-aging composition comprising a suitable amount of each of the following:
a) water,
b) niacinamide,
c) propylene glycol,
d) glycerin,
e) butylene glycol,
f) tranexamic acid,
g) rumex occidentalis extract,
h) ascorbic acid,
i) acetyl tyrosine,
j) aminopropyl ascorbyl phosphate,
k) sxifraga sarmentosa extract,
l) arbutin,
m) paeonia suffruticosa extract,
n) scutellaria baicalensis root extract,
o) glutathione,
p) hydroxyethylcellulose (HEC),
q) citric acid,
r) sodium sulfite,
s) disodium EDTA,
t) phenoxyethanol, and
u) ethylhexylglycerin.

The present invention provides a combination of the following:
(a) a refining composition and a brightening composition;
(b) an anti-scaring composition and a brightening composition; and
(c) a firming and an anti-aging composition.

The present invention provides a method of increasing skin brightness of an individual, wherein the method comprises applying the individual's skin an effective amount of a brightening composition and refining composition, or ani-scaring composition and brightening composition, or firming composition and anti-aging composition.

The present invention provides a method of increasing skin firmness of an individual, wherein the method comprises applying the individual's skin an effective amount of a brightening composition and refining composition, or ani-scaring composition and brightening composition, or firming composition and anti-aging composition.

The present invention provides a method of increasing skin smoothness of an individual, wherein the method comprises applying the individual's skin an effective amount of a brightening composition and refining composition, or ani-scaring composition and brightening composition, or firming composition and anti-aging composition.

The present invention provides a method of decreasing in fine lines/wrinkle of an individual, wherein the method comprises applying the individual's skin an effective amount of a brightening composition and refining composition, or ani-scaring composition and brightening composition, or firming composition and anti-aging composition.

The present invention provides a method of fading melasma/hyperpigmentation of an individual, wherein the method comprises applying the individual's skin an effective amount of a brightening composition and refining composition, or ani-scaring composition and brightening composition, or firming composition and anti-aging composition.

The present invention provides a method of fading/shallowing facial marks of an individual, wherein the method comprises applying the individual's skin an effective amount of a brightening composition and refining composition, or ani-scaring composition and brightening composition, or firming composition and anti-aging composition.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a series of color photographs depicting comparative pictures of a first person's front facial view, right-side facial view and left-side facial view illustrating skin improvement after using MESO Bright and MESO Refine formulation over a duration of three, six and nine weeks.

FIG. 2 depicts a series of color photographs depicting comparative pictures of a second person's front facial view, right-side facial view and left-side facial view illustrating skin improvement after using MESO Bright and MESO Refine formulation over a duration of three, six and nine weeks.

FIG. 3 depicts a series of color photographs depicting comparative pictures of a third person's front facial view, right-side facial view and left-side facial view illustrating skin improvement after using MESO Bright and MESO Refine formulation over a duration of three, six and nine weeks.

FIG. 4 depicts a series of color photographs depicting comparative pictures of a fourth person's front facial view, right-side facial view and left-side facial view illustrating skin improvement after using MESO Bright and MESO Refine formulation over a duration of three, six and nine weeks.

FIG. 5 depicts a series of color photographs depicting comparative pictures of a fifth person's front facial view, right-side facial view and left-side facial view illustrating skin improvement after using MESO Bright and MESO Refine formulation over a duration of three and six weeks.

FIG. 6 depicts a series of color photographs depicting comparative pictures of a sixth person's front facial view, right-side facial view and left-side facial view illustrating skin improvement after using MESO Scar and MESO Bright formulation over a duration of three, six and nine weeks.

FIG. 7 depicts a series of color photographs depicting comparative pictures of a seventh person's front facial view, right-side facial view and left-side facial view illustrating skin improvement after using MESO Scar and MESO Bright formulation over a duration of three, six and nine weeks.

FIG. 8 depicts a series of color photographs depicting comparative pictures of an eighth person's front facial view, right-side facial view and left-side facial view illustrating skin improvement after using MESO Scar and MESO Bright formulation over a duration of three, six and nine weeks.

FIG. 9 depicts a series of color photographs depicting comparative pictures of a ninth person's front facial view, right-side facial view and left-side facial view illustrating skin improvement after using MESO Scar and MESO Bright formulation over a duration of three, six and nine weeks.

FIG. 10 depicts a series of color photographs depicting comparative pictures of a tenth person's front facial view, right-side facial view and left-side facial view illustrating skin improvement after using MESO Scar and MESO Bright formulation over a duration of three, six and nine weeks.

FIG. 11 depicts a series of color photographs depicting comparative pictures of an eleventh person's front facial view, right-side facial view and left-side facial view illustrating skin improvement after using MESO Scar and MESO Bright formulation over a duration of three, six and nine weeks.

FIG. 12 depicts a series of color photographs depicting comparative pictures of a twelfth person's front facial view, right-side facial view and left-side facial view illustrating skin improvement after using MESO Scar and MESO Bright formulation over a duration of three weeks.

FIG. 13 depicts a series of color photographs depicting comparative pictures of a thirteen person's front facial view, right-side facial view and left-side facial view illustrating skin improvement after using MESO Firming and MESO Darkspot formulation over a duration of three, six and nine weeks.

FIG. 14 depicts a series of color photographs depicting comparative pictures of a fourteen person's front facial view, right-side facial view and left-side facial view illustrating skin improvement after using MESO Firming and MESO Darkspot formulation over a duration of three, six and nine weeks.

FIG. 15 depicts a series of color photographs depicting comparative pictures of a fifteen person's front facial view, right-side facial view and left-side facial view illustrating skin improvement after using MESO Firming and MESO Darkspot formulation over a duration of three, six and nine weeks.

FIG. 16 depicts a series of color photographs depicting comparative pictures of a sixteenth person's front facial view, right-side facial view and left-side facial view illustrating skin improvement after using MESO Firming and MESO Darkspot formulation over a duration of three, six and nine weeks.

### DETAILED DESCRIPTION

### Definition

Unless defined otherwise, all technical and scientific terms used herein have the meaning commonly understood by persons of ordinary skill in the art to which this subject matter pertains.

The term “a” or “an”, as used herein, includes the singular and the plural, unless specifically stated otherwise. Therefore, the terms “a,” “an,” or “at least one” can be used interchangeably in this application.

Throughout the application, descriptions of various embodiments use the term “comprising”; however, it will be understood by one of skill in the art, that in some specific instances, an embodiment can be described using the language “consisting essentially of” or “consisting of.”

The term “about” herein specifically includes ±10% from the indicated values in the range. In addition, the endpoints of all ranges directed to the same component or property herein are inclusive of the endpoints, are independently combinable, and include all intermediate points and ranges.

As used herein, the term “about” when used in connection with a numerical value includes ±10% from the indicated value. In addition, all ranges directed to the same component or property herein are inclusive of the endpoints, are independently combinable, and include all intermediate points and ranges. It is understood that where a parameter range is provided, all integers within that range, and tenths thereof, are also provided by the invention. For example, “30-45%” includes 30%, 30.1%, 30.2%, etc. up to 45%.

“Microneedling” is a cometic procedure that is used to encourage collagen production using small, sterilized needles. It may help smooth, firm, and tone skin and improve the appearance of scars, acne, and wrinkles.

“Dermaroller” and “Dermapen” is a tool in which tiny little needles are inserted into the surface of the skin via a rolling or stamping device. Dermarolling works by creating microscopic wounds that induce collagen and elastin production. Collagen is the most abundant protein found in the human body and is responsible for holding together connective tissue like skin, muscles, tendons, cartilage, and bones. It's believed that collagen production slows down as you age, which translates to skin thinning and impaired barrier function. The recovery process does depend largely on the length of the needles used. The longer the needles, the deeper the wound—and that means the longer the recovery time.

“Dermarollers” use a rolling head of hundreds of stainless steel needles to prick the skin. They're typically used on wide areas like the face, neck, and abdominal area to treat wrinkles, acne scars, dark circles under the eyes, and other fine lines. Usually, they're combined with other skincare products like hvaluronic acid. Derma rollers are an excellent choice if the patient only needs light treatment. They're best suited for stretch marks and other light wrinkles that don't need a lot of product or treatment. Combined with the right skincare product, using derma rollers can give the skin overall improvements.

“Dermapens” are handheld devices that are similar to “Dennarollers”, but they work via oscillating pulses that quickly move the needle up and down the treatment site. Unlike rollers that move horizontally across the skin, Derma Pens move vertically. They're more maneuverable than Dermarollers since they're easier to grip. “Dermapens” are an excellent choice for many professional procedures since it improves the results while reducing the common drawbacks of using a derma roller. For this reason, they're often limited to clinical and cosmetic practices, though there are a few commercial models that patients can buy and use at home.

As used herein a “solvent” is a substance used to dissolve other ingredients, such as pigments, oils, or active compounds, to create a stable and homogenous product. Solvents play a crucial role in formulating cosmetic products by facilitating the blending of different components and ensuring the desired consistency, texture, and performance of the final product. They can be organic or inorganic compounds, and their selection depends on factors such as the solubility of the ingredients, desired product properties, safety considerations, and regulatory requirements.

Common solvents used in cosmetic products include: Water Water is used as a solvent in cosmetics, particularly in aqueous formulations like lotions, creams, and gels. It can dissolve water-soluble ingredients and serves as a base for many cosmetic products. Alcohols: Ethanol (alcohol) and other alcohols like isopropyl alcohol are used as solvents in cosmetics to dissolve both water-soluble and oil-soluble ingredients. They are often found in toners, astringents, and some hair care products Glycerin: Glycerin is a versatile solvent that is both water-soluble and can dissolve many substances that are not soluble in water. It is commonly used in moisturizers, serums, and other skincare products. Silicones. Silicone-based solvents like cyclomethicone and dimethicone are used in cosmetics to dissolve silicone-based ingredients, improve product spreadability, and impart a silky texture to formulations Oils: Various oils such as mineral oil, jojoba oil, and vegetable oils are used as solvents in cosmetic products, especially in makeup removers, cleansers, and emollient-rich formulations Esters: Esters are compounds derived from organic acids and alcohols. They are used as solvents in cosmetics due to their ability to dissolve a wide range of ingredients while providing emollient and conditioning properties. Propylene Glycol: Propylene glycol is a solvent used in skmcare and hair care products. It helps dissolve ingredients and can also fimction as a humectant, attracting moisture to the skin or hair. Solvent Blends: Sometimes, cosmetic formulators use a combination of solvents to achieve specific formulation goals. These solvent blends can offer a balance of solubility, viscosity, and sensory properties.

As used herein, a “chelating agent” also known as a sequestering agent or chelator, is a substance used in cosmetic products to bind and neutralize metal ions present in water, formulations, or on the skin. These metal ions, such as calcium, magnesium, iron, and copper, can catalyze reactions that lead to product degradation, color changes, rancidity, or reduced efficacy of active ingredients. Chelating agents help prevent these undesirable effects by forming stable complexes with metal ions, keeping them inactive and improving the overall stability and performance of cosmetic formulations.

A “chelator” as used herein, is a compound that is able to chelate or electrostatically interact with a range of transition metal ions (Fe, Cu, Co, Cr & Mn) which can facilitate and catalyze decomposition via oxidation. They are usually used in combination with an antioxidant. Examples of such chelators are Ethylene Diamine Tetra-acetic acid (EDTA) and its sodium salts, sodium pyrophosphate, Citric acid, Gluconic acid, Catechol and various thiol derivatives.

As used herein. “antioxidant agents” in cosmetic products are substances that help protect skin and cosmetic formulations from oxidative damage caused by free radicals. Free radicals are highly reactive molecules that can be generated by various factors such as UV radiation, pollution, and metabolic processes. When free radicals interact with skin cells or cosmetic ingredients, they can lead to oxidative stress, premature aging, inflammation, and damage to proteins, lipids, and DNA. Antioxidants work by neutralizing free radicals, thus preventing or minimizing the harmful effects of oxidative stress. In cosmetics, antioxidant agents play a crucial role in promoting skin health, maintaining product stability, and preserving the efficacy of active ingredients. They are often included in skincare formulations, sunscreens, hair care products, and other cosmetic products.

An “antioxidant,” as used herein, is a substance that inhibits oxidation of chemical compounds. Examples of typical antioxidants include alpha tocopherol (all isomers), beta tocopherol, delta tocopherol, gamma tocopherol, tocopherols, ascorbic acid, ascorbyl palmitate, butylated hydroxyanisol (BHA), butylated hydroxytoluene (BHT), fumaric acid, malic acid, methionine, propyl gallate, sodium ascorbate, sodium metabisulfate, sodium thiosulfate, sodium bisulfate. The chemical compounds protected from oxidation by an antioxidant include active pharmaceutical ingredients (API) and excipients containing moieties susceptible to oxidation.

As used herein, “preservatives” in cosmetic products are substances or mixtures of substances that are added to formulations to prevent or inhibit microbial growth, thereby extending the shelf life and maintaining the safety and stability of the product. Preservatives play a crucial role in cosmetics by protecting against microbial contamination, which can lead to product spoilage, degradation, and potential health risks for consumers.

As used herein. “skin-conditioning agents” in cosmetic products refer to substances or ingredients that are formulated to improve the appearance, texture, and overall health of the skin. These agents play a vital role in skincare formulations by hydrating, moisturizing, softening, and nourishing the skin, thereby enhancing its smoothness, elasticity, and comfort.

As used herein, “humectants” in cosmetic products are substances or ingredients that attract moisture from the surrounding environment or deeper layers of the skin, helping to increase and maintain hydration levels. These agents play a crucial role in skincare formulations by improving skin moisture retention, enhancing skin elasticity, and creating a smoother and more supple appearance.

A “humectant,” as used herein, is an excipient that can increase the water level in the upper layers of the skin. Examples of humectants approved for use in topical drug products by the FDA include, but are not limited to, the following: hexylene glycol, propylene glycol, sorbitol, lactic acid, sodium lactate, methyl gluceth-1O, methyl gluceth-20, and polyethylene glycols.

As used herein, “thickening agents” in cosmetic products are substances or ingredients that are added to formulations to increase viscosity, enhance texture, and improve the consistency of the product. These agents play a crucial role in cosmetics by providing the desired thickness, stability, and spreadability to various formulations such as creams, lotions, gels, serums, and hair care products.

As used herein, a “dispersing agent”, also known as a dispersant or dispersible polymer, is a substance or ingredient used in cosmetic products to facilitate the dispersion of insoluble or poorly soluble substances (such as pigments, minerals, or active ingredients) in liquid or semi-solid formulations. Dispersing agents play a crucial role in cosmetics by ensuring even distribution, stability, and uniform coloration of products such as lotions, creams, makeup, and sunscreens.

Invasive” cosmetic treatment means the treatment requires breaking of the skin,

“Noninvasive” cosmetic treatment means the treatment doesn't require breaking of the skin.

There are different skin conditions described in the application.

Acne Vulgaris: A common skin condition characterized by the formation of comedones (blackheads and whiteheads), papules, pustules, and sometimes nodules or cysts. Acne is often associated with oily skin, hormonal changes, and inflammation of hair follicles.

Eczema (Atopic Dermatitis): Eczema is a chronic inflammatory skin condition that causes red, itchy, and inflamed patches of skin. It can be triggered by various factors such as genetics, environmental allergens, irritants, and immune system dysfunction.

Psoriasis: Psoriasis is a chronic autoimmune skin disorder characterized by the rapid growth of skin cells, leading to thick, scaly, and often itchy patches or plaques. It can occur on the scalp, elbows, knees, and other areas of the body.

Rosacea: Rosacea is a chronic inflammatory skin condition that primarily affects the face, causing redness, flushing, visible blood vessels, and sometimes papules and pustules resembling acne. It may also involve ocular symptoms in some cases.

Hyperpigmentation: Hyperpigmentation refers to darkening of the skin due to an overproduction of melanin. It can result from sun exposure, hormonal changes (melasma), post-inflammatory processes, or certain medications.

Hypopigmentation: Hypopigmentation is the loss of skin color, leading to lighter or white patches on the skin. It can be caused by conditions such as vitiligo, post-inflammatory changes, or certain genetic factors.

Seborrheic Dermatitis. Seborrheic dermatitis is a common skin condition characterized by red, greasy, and scaly patches, particularly in areas rich in sebaceous glands such as the scalp (dandruff), face (eyebrows, sides of the nose), and upper chest.

Contact Dermatitis: Contact dermatitis is an inflammatory skin reaction caused by contact with irritants (irritant contact dermatitis) or allergens (allergic contact dermatitis). It presents as redness, itching, and sometimes blisters or rash at the site of contact.

Fungal Infections. Fungal skin infections such as ringworm (tinea corporis), athlete's foot (tinea pedis), and fungal nail infections (onychomycosis) are caused by fungal organisms and result in itching, redness, scaling, and discomfort.

Actinic Keratosis: Actinic keratosis is a precancerous skin condition caused by long-term sun exposure, leading to rough, scaly, and sometimes pigmented patches or lesions on sun-exposed areas, particularly in fair-skinned individuals.

There are different skin types described in the application. Normal skin is well-balanced, with few imperfections and a smooth texture. Dry skin lacks moisture, often feeling tight and rough, and may experience flakiness or redness. Oily skin produces excess sebum, leading to a shiny appearance and is prone to acne and enlarged pores. Combination skin has both oily and dry areas, with the T-zone usually oily and the cheeks dry or normal. Sensitive skin reacts easily to products or environmental factors, requiring gentle, fragrance-free care to avoid irritation.

The terms “treat(s)”, “treating”, “treated”, and “treatment” as used herein include preventative (e.g., prophylactic), ameliorative, palliative and curative uses and/or results. The terms preventative or prophylactic are used interchangeably and refer to treatment prior to the onset of one or more signs or symptoms of a particular condition or disease state. More specifically, these terms refer to the treatment of patients that are largely asymptomatic, i.e. where symptoms of a particular condition or disease state are not readily apparent or detectable, and which results in the substantial prevention, suppression or delay in the onset of one or more signs or symptoms of a particular condition or disease state. An ameliorative treatment is one that improves and/or lessens the severity of one or more signs or symptoms of a particular condition or disease state.

The phrases “therapeutic” and “therapeutically effective amount” as used herein respectively denote an effect and an amount of a compound, composition or medicament that (a) treats a particular disease, condition or disorder; (b) attenuates, ameliorates or eliminates one or more signs, symptoms of or complications arising from a particular disease, condition or disorder; (c) prevents or delays the onset of one or more signs, symptoms of or complications associated with a particular disease, condition or disorder. It should be understood that the terms “therapeutic” and “therapeutically effective amount” encompass any one of the aforementioned effects (a)-(c), either alone or in combination with any of the others (a)-(c). The terms “mammal”, “patient” and “subject” refer to warm blooded animals such as, for example, guinea pigs, mice, rats, gerbils, cats, rabbits, dogs, monkeys, chimpanzees, and humans. The “therapeutically effective amount” will vary depending on the composition, the compound, the therapy, the course of treatment, the disease, disorder, or condition, and its severity and the age, weight, etc., of the subject to be treated.

As used herein, the term “therapeutically effective amount” refers to an amount of the API which is sufficient to reduce or ameliorate the severity, duration, progression, or onset of a disorder being treated, prevent the advancement of a disorder being treated, cause the regression of, prevent the recurrence, development, onset or progression of a symptom associated with a disorder being treated, or enhance or improve the prophylactic or therapeutic effect(s) of another therapy. The precise amount of compound administered to a subject will depend on the type and severity of the disease or condition and on the characteristics of the subject, such as general health, age, sex, body weight and tolerance to drugs. It will also depend on the degree, severity and type of the disorder being treated. The skilled artisan will be able to determine appropriate dosages depending on these and other factors. When co-administered with other agents, e. g., when co-administered with anti-infectives such as Benzoyl Peroxide or Vitamin A derivatives such as Retinoic acid, a “therapeutically effective amount” of the second agent will depend on the type of drug used. Suitable dosages are known for approved agents and can be adjusted by the skilled artisan according to the condition of the subject, the type of condition(s) being treated and the amount of a compound of the invention being used. In cases where no amount is expressly noted, an effective amount should be assumed.

As used herein, the terms “treat”, “treatment” and “treating” refer to the reduction or amelioration of the progression, severity and/or duration of a disorder being treated, or the amelioration of one or more symptoms (preferably, one or more discernible symptoms) of a disorder being treated resulting from the administration of one or more pharmaceutical compositions of the present invention. In specific embodiments, the terms “treat”, “treatment” and “treating” refer to the amelioration of at least one measurable physical parameter being treated. For example, for acne the total count of skin lesions and/or the count of skin pustules and associated inflammatory area under accepted Dermatological parameters will be used to measure the treatment effectiveness. In other embodiments the terms “treat”, “treatment” and “treating” refer to the inhibition of the progression of a disorder being treated, either physically by, e.g., stabilization of a discernible symptom, physiologically by, e.g., stabilization of a physical parameter, or both. In the embodiments in which the disorder being evaluated

An “ointment,” as used herein, is a semisolid dosage form, usually containing less than 20% Water and volatiles and more than 50% hydrocarbons, waxes, or polyols as the vehicle. This dosage form is generally for external application to the skin or mucous membranes.

A “semisolid,” as used herein refers to the aggregate state of matter which appears a liquid but which is not pourable; it does not flow or conform to its container shape at room temperature nor does it flow at low shear stress and generally exhibits plastic flow behavior.

A “gel,” as used herein, is a semisolid dosage form that contains a gelling agent to thicken a solution or fine particle dispersion. The formulation can appear as a transparent or translucent fluid in a container, depending on how much fine particle dispersion is incorporated. Thickening the formulation aids in topical application and adherence to the skin in order to aid diffusion and film formation at the skin surface and to provide a matrix from which the water and nonaqueous solvents can exert their emollient, humectant, solubilization and permeation enhancement properties which are jointly responsible for the measured drug delivery to the skin tissues at specific sites on the body.

A “nonaqueous” gel as used herein, is a semisolid dosage form that contains a gelling agent to thicken a solution or fine particle dispersion but which is either completely comprised of nonaqueous solvents or with a minimal amount of water, typically less than 10% by weight or volume. These gels will generally solubilize higher concentrations of more lipophilic drugs than the aqueous gels.

A “cream” is a biphasic dispersion or emulsion that has an aqueous phase and an oil phase whereby significant energy and the use of surface active agents and stabilizers are required in order to prevent coalescence and separation. A Cream is typically a semisolid, thickened by the use of previously described gelling agents and long chain lipids used in the oil phase. The API if highly lipid soluble will tend to be located in the oil phase or solubilized by surfactants in the aqueous phase. Absorption at the skin surface is facilitated by the excellent cosmesis and spreadability that creams afford and the delivery of an oil rich film with high concentrations of drug once the aqueous phase has evaporated or been absorbed at the skin surface.

A “foam,” as used herein, is a dosage form containing gas bubbles dispersed in a liquid that contains less than 50% Water, and several nonaqueous solvents and surfactants that cause rapid dispersion and film forming on the skin to facilitate coverage over the area of skin to be treated and rapid absorption.

As used herein, the term “skin” refers to the outer covering of the body. In humans, it is the largest organ of the integumentary system. The skin has multiple layers of ectodermal tissue and guards the underlying muscles, bones, ligaments and internal organs. Human skin is similar to that of most other mammals, except that it is not protected by a fur. Though nearly all human skin is covered with hair follicles, it can appear hairless. There are two general types of skin, hairy and glabrous skin. The adjective cutaneous means “of the skin” (from Latin cutis, skin).

As used herein, the term “acne” refers to acne vulgaris, a common human skin disease, characterized by areas of skin with comedones (blackheads and whiteheads), papules (pinheads), nodules (large papules), pimples, and possibly scarring. Acne affects mostly skin with the densest population of sebaceous follicles; these areas include the face, the upper part of the chest, and the back. Severe acne is inflammatory, but acne can also manifest in non-inflammatory forms. Severe acne also includes the condition known as ‘nodulocystic acne’. Acne lesions are caused by changes in pilosebaceous units, skin structures consisting of a hair follicle and its associated sebaceous gland, changes that require androgen stimulation.

### Refining Composition

The current invention provides a refining composition comprising a suitable amount of each of the following:
a) sh-Oligopeptide-1,
b) water,
c) glycerin,
d) niacinamide,
e) propylene glycol,
f) laminaria saccharina extract,
g) starfish coelomic fluid extract,
h) hydroxyethylcellulose (HEC),
i) disodium ethylenediaminetetraacetic acid (EDTA),
j) phenoxyethanol, and
k) ethylhexylglycerin.

Preferably, the present invention provides a refining composition comprising a suitable amount of each of the following:
a) Glycerin and Laminaria saccharina extract;
b) Niacinamide;
c) sh-oligopeptide-1; and
d) Glycerin (and) Starfish Coelomic Fluid Extract.

More preferably, the present invention provides a refining composition comprising a suitable amount of each of the following:
a) Glycerin and Laminaria saccharina extract at a weight percentage of 2.50%;
b) Niacinamide at a weight percentage of 1.50%;
c) sh-oligopeptide-1 at a weight percentage of 54%; and
d) Glycerin and Starfish Coelomic Fluid Extract at a weight percentage of 1%.

Most preferably, the present invention provides a refining composition comprising a suitable amount of ingredient at weight percentage recited in Table 1.

TABLE 1
REFINING COMPOSITION (MESO REFINE)

| RAW MATERIAL | | | |
|---|---|---|---|
| NAME | INCI NAME | FUNCTION | % |
| Water | Aqua | Solvent | 37.70% |
| HEC | Hydroxyethylcellulose | Thickener | 1.00% |
| Glycerin | Glycerin | Humectant | 0.40% |
| Propylene Glycol | Propylene glycol | Humectant | 1.00% |
| EDTA | Disodium EDTA | Chelating Agent | 0.10% |
| VivenSea ™ | Glycerin (and) Laminaria | Anti-Aging, Soothing, improved | 2.50% |
| | saccharina extract | skin elasticity | |
| Niacinamide | Niacinamide | Anti-Aging, Pore Minimizer, | 1.50% |
| | | Soothing, anti-inflammatory, | |
| | | skin brightening, | |
| Bio-NanoEGF1 | sh-oligopeptide-1 | Anti-Aging, Hydration, | 54.00% |
| | | Soothing, skin rejuvenation, | |
| | | anti-aging, wrinkle prevention, | |
| | | wound healing, regeneration of | |
| | | the epidermis | |
| Juventide ® | Glycerin (and) Starfish | Anti-Aging, Hydration, | 1.00% |
| | Coelomic Fluid Extract | Soothing, anti-wrinkle, improve | |
| | | skin elasticity and firmness | |
| Nipaguard EHP ® | Phenoxyethanol & | Preservative | 0.80% |
| | Ethylhexylglycerin | | |

| | |
|---|---|
| TOTAL | 100.00% |

In some embodiments, the weight percentage of glycerin and laminaria saccharina extract is between about 0.5% to 4.5%.

In some embodiments, the weight percentage of glycerin and laminaria saccharina extract is between about 0.5% to 2.5%.

In some embodiments, the weight percentage of glycerin and laminaria saccharina extract is between about 1% to 2.5%.

In some embodiments, the weight percentage of glycerin and laminaria saccharina extract is between about 1.5% to 2.5%.

In some embodiments, the weight percentage of glycerin and laminaria saccharina extract is between about 2% to 2.5%.

In some embodiments, the weight percentage of glycerin and laminaria saccharnna extract is 2.5%.

In some embodiments, the weight percentage of glycerin is between 0.1-0.3%.

In some embodiments, the weight percentage of niacinamiide is between about 0.5% to 2.5%.

In some embodiments, the weight percentage of niacinamiide is between about 0.5% to 1.5%.

In some embodiments, the weight percentage of niacinamnide is between about 1% to 1.5%.

In some embodiments, the weight percentage of niacinamide is 1.5%.

In some embodiments, the weight percentage of sh-Oligopeptide-1 is between about 30-70%.

In some embodiments, the weight percentage of sh-Oligopeptide-1 is between about 40-70%.

In some embodiments, the weight percentage of sh-Oligopeptide-1 is between about 50-70%.

In some embodiments, the weight percentage of sh-Oligopeptide-1 is between about 50-60%.

In some embodiments, the weight percentage of sh-Oligopeptide-1 is 54%.

In some embodiments, the weight percentage of glycerin (and) starfish coelomic fluid extract is between about 0.2% to 2%.

In some embodiments, the weight percentage of glycerin (and) starfish coelomic fluid extract is between about 0.4% to 1.8%.

In some embodiments, the weight percentage of glycerin (and) starfish coelomic fluid extract is between about 0.6% to 1.6%.

In some embodiments, the weight percentage of glycerin (and) starfish coelomic fluid extract is between about 0.8% to 1.4%.

In some embodiments, the weight percentage of glycerin (and) starfish coelomic fluid extract is between about 1% to 1.2%.

In some embodiments, the weight percentage of glycerin (and) starfish coelomic fluid extract is 1%.

In some embodiments, the weight percentage of glycerin is 0.4%.

In some embodiments, the weight percentage of HEC is between about 0.5 to 1.5%.

In some embodiments, the weight percentage of HEC is 1%.

In some embodiments, the weight percentage of propylene glycol is between about 0.5 to 1.5%.

In some embodiments, the weight percentage of propylene glycol is 1%.

In some embodiments, the weight percentage of disodium EDTA is between about 0.05-0.15%.

In some embodiments, the weight percentage of disodium EDTA is 0.1%.

In some embodiments, the weight percentage of phenoxyethanol and ethylhexylglycerin is between about 0.4-1.2%.

In some embodiments, the weight percentage of phenoxyethanol and ethylhexylglycerin is 0.8%.

In some embodiments, the weight percentage of water is between 30-40%.

In some embodiments, the weight percentage of water is 37.7%.

In some embodiments, the current invention provides a composition, wherein
a) the weight percentage of glycerin and laminaria saccharina extract is between about 1.5% to 5%;
b) the weight percentage of niacinamide is between about 0.5% to 5%;
c) the weight percentage of sh-Oligopeptide-1 is between about 40-80%; and
d) the weight percentage of glycerin (and) starfish coelomic fluid extract is between about 0.1% to 10%.

In some embodiments, the current invention provides a composition, wherein
a) the weight percentage of glycerin and laminaria saccharina extract is 2.5%;
b) the weight percentage ofniacinamide is 1.5%;
c) the weight percentage of sh-Oligopeptide-1 is 54%;
d) the weight percentage of glycerin (and) starfish coelomic fluid extract is 1%;
e) the weight percentage ofHEC is 1%;
f) the weight percentage ofdisodium EDTA is between about 0.1%; and
g) the weight percentage of phenoxyethanol and ethylhexylglycerin is between about 0.8%; and
h) the weight percentage of water is 37.7%.

### Brightening Composition

The current invention provides a brightening composition comprising a suitable amount of each of the following:
a) water,
b) glycerin,
c) propylene glycol,
d) kojic acid,
e) glycolic acid,
f) potassium azelaoyl diglycinate,
g) sodium salt of pyroglutamic acid (sodium PCA),
h) arctostaphylos uva-ursi leaf extract,
i) magnesium ascorbyl phosphate,
j) alpha-arbutin,
k) hydroxyethylcellulose (HEC),
l) sodium benzoate,
m) sodium metabisulfite,
n) disodium ethylenediaminetetraacetic acid (EDTA),
o) phenoxyethanol, and
p) ethylhexylglycerin.

Preferably, the present invention provides a brightening composition comprising a suitable amount of each of the following:
a) alpha arbutin;
b) arctostaphylos uva-ursi leaf extract, magnesium ascorbyl phosphate, glycerin, water, sodium benzoate, sodium metabisulfite, and phenoxyethanol; and
c) kojic acid, glycolic acid, potassium azelaoyl diglycinate, Sodium PCA, and glycerin.

More preferably, the present invention provides a brightening composition comprising a suitable amount of each of the following:
a) alpha arbutin at a weight percentage of 0.2%;
b) arctostaphylos uva-ursi leaf extract, magnesium ascorbyl phosphate, glycerin, water, sodium benzoate, sodium metabisulfite, and phenoxyethanol at a weight percentage of 5%; and
c) kojic acid, glycolic acid, potassium azelaoyl diglycinate, Sodium PCA, and glycerin at a weight percentage of 2%.

Most preferably, the present invention provides a brightening composition of a formulation comprising a suitable amount of ingredient at weight percentage recited in Table 2.

TABLE 2
BRIGHTENING COMPOSITION (MESO BRIGHT)

| RAW MATERIAL | | | |
|---|---|---|---|
| NAME | INCI NAME | FUNCTION | % |
| Water | Aqua | Solvent | 89.50% |
| HEC | Hydroxyethylcellulose | Thickener | 0.40% |
| Glycerin | Glycerin | Humectant | 1.00% |
| Propylene Glycol | Propylene glycol | Humectant | 1.00% |
| EDTA | Disodium EDTA | Chelating Agent | 0.10% |
| Alpha-Arbutin | Alpha-Arbutin | Brightening, potent skin whitening | 0.20% |
| Melfade ® PF | Arctostaphylos Uva-Ursi Leaf | Brightening, Antioxidant, | 5.00% |
| | Extract (and) Magnesium | combination of a multi-function | |
| | Ascorbyl Phosphate (and) | vitamin C derivative and a highly | |
| | Glycerin (and) Aqua (and) | purified extract of bearberry leaves | |
| | Sodium Benzoate (and) Sodium | (Arctostaphylos uva-ursi), known | |
| | Metabisulfite (and) | as a natural source of arbutin. | |
| | Phenoxyethanol | MELFADE ® PF is a very effective | |
| | | inhibitor of tyrosinase and has | |
| | | strong antioxidant properties. | |
| Azeglyjic ® Optima | Kojic Acid (And) Glycolic Acid | Brightening, skin whitening, anti- | 2.00% |
| | (And) Potassium Azeloyl | aging, moisturizing | |
| | Diglycinate (And) Sodium PCA | | |
| | (And) Glycerin | | |
| Nipaguard ® EHP | Phenoxyethanol (and) | Preservative | 0.80% |
| | Ethylhexylglycerin | | |

| | |
|---|---|
| TOTAL | 100.00% |

In some embodiments, the weight percentage of alpha arbutin is between about 0.05% to 0.6%.

In some embodiments, the weight percentage of alpha arbutin is between about 0.05% to 0.5%.

In some embodiments, the weight percentage of alpha arbutin is between about 0.05% to 0.4%.

In some embodiments, the weight percentage of alpha arbutin is between about 0.1% to 0.3%.

In some embodiments, the weight percentage of alpha arbutin is 0.2%.

In some embodiments, the weight percentage of arctostaphylos uva-ursi leaf extract, magnesium ascorbyl phosphate, glycerin, water, sodium benzoate, sodium metabisulfite, and phenoxyethanol is between about 1% to 10%.

In some embodiments, the weight percentage of arctostaphylos uva-ursi leaf extract, magnesium ascorbyl phosphate, glycerin, water, sodium benzoate, sodium metabisulfite, and phenoxyethanol is between about 2% to 9%.

In some embodiments, the weight percentage of arctostaphylos uva-ursi leaf extract, magnesium ascorbyl phosphate, glycerin, water, sodium benzoate, sodium metabisulfite, and phenoxyethanol is between about 3% to 8%.

In some embodiments, the weight percentage of arctostaphylos uva-ursi leaf extract, magnesium ascorbyl phosphate, glycerin, water, sodium benzoate, sodium metabisulfite, and phenoxyethanol is between about 4% to 7%.

In some embodiments, the weight percentage of arctostaphylos uva-ursi leaf extract, magnesium ascorbyl phosphate, glycerin, water, sodium benzoate, sodium metabisulfite, and phenoxyethanol is between about 4% to 6%.

In some embodiments, the weight percentage of arctostaphylos uva-ursi leaf extract, magnesium ascorbyl phosphate, glycerin, water, sodium benzoate, sodium metabisulfite, and phenoxyethanol is 5%.

In some embodiments, the weight percentage of kojic acid, glycolic acid, potassium azelaoyl diglycinate, Sodium PCA, and glycerin is between about 0.5-5%.

In some embodiments, the weight percentage of kojic acid, glycolic acid, potassium azelaoyl diglycinate, Sodium PCA, and glycerin is between about 0.54%.

In some embodiments, the weight percentage of kojic acid, glycolic acid, potassium azelaoyl diglycinate, Sodium PCA, and glycerin is between about 0.5-3%.

In some embodiments, the weight percentage of kojic acid, glycolic acid, potassium azelaoyl diglycinate, Sodium PCA, and glycerin is between about 1-3%.

In some embodiments, the weight percentage of kojic acid, glycolic acid, potassium azelaoyl diglycinate, Sodium PCA, and glycerin is between about 1.5-2.5%.

In some embodiments, the weight percentage of kojic acid, glycolic acid, potassium azelaoyl diglycinate, Sodium PCA, and glycerin is 2%.

In some embodiments, wherein the weight percentage of HEC is between about 0.1 to 0.8%.

In some embodiments, the weight percentage of HEC is between about 0.2 to 0.7%.

In some embodiments, the weight percentage of HEC is between about 0.3 to 0.6%.

In some embodiments, the weight percentage of HEC is 0.4%.

In some embodiments, the weight percentage of glycerin is between about 0.5 to 1.5%.

In some embodiments, the weight percentage of glycerin is 1%.

In some embodiments, the weight percentage of phenoxyethanol and ethylhexylglycerin is between about 0.6 to 1%.

In some embodiments, the weight percentage of phenoxyethanol and ethylhexylglycerin is 0.8%.

In some embodiments, the weight percentage of propylene glycol is 1%.

In some embodiments, the weight percentage of disodium EDTA is between about 0.05 to 0.15%.

In some embodiments, the weight percentage of disodium EDTA is between 0.08-0.12%.

In some embodiments, the weight percentage of disodium EDTA is 0.1%.

In some embodiments, the weight percentage of water is between 85-95%.

In some embodiments, the weight percentage of water is between about 89 and 90%.

In some embodiments, the weight percentage of water is 89.5%.

In some embodiments, the current invention provides a composition wherein the weight percentage of
a) alpha arbutin is between about 1.5% to 5%;
b) arctostaphylos uva-ursi leaf extract, magnesium ascorbyl phosphate, glycerin, water, sodium benzoate, sodium metabisulfite, and phenoxyethanol is between about 3% to 7%; and
c) kojic acid, glycolic acid, potassium azelaoyl diglycinate. Sodium PCA, and glycerin is between about 2-6%.

In some embodiments, the current invention provides a composition wherein the weight percentage of
a) alpha arbutin is 0.2%;
b) arctostaphylos uva-ursi leaf extract, magnesium ascorbyl phosphate, glycerin, water, sodium benzoate, sodium metabisulfite, and phenoxyethanol is 5%;
c) kojic acid, glycolic acid, potassium azelaoyl diglycinate, Sodium PCA, and glycerin is 2%;
d) HEC is 0.4%;
e) glycerin is 1%;
f) propylene glycol is 1%;
g) disodium EDTA is 0.1%;
h) water is 89.5%; and
i) phenoxyethanol and ethylhexylglycerin is 0.8%.

### Anti-Scarring Composition

The present invention provides an anti-scarring composition comprising a suitable amount of each of the following:
a) sh-oligopeptide-1,
b) water,
c) snail secretion filtrate,
d) propylene glycol,
e) glycerin,
f) hydroxyethylcellulose (HEC),
g) copper tripeptide-1,
h) phenoxyethanol, and
i) ethylhexylglycerin.

Preferably, the present invention provides an anti-scarring composition comprising a suitable amount of each of the following:
a) snail secretion filtrate;
b) copper tripeptide-1; and
c) sh-oligopeptide-1.

More preferably, the present invention provides an anti-scarring composition of a formulation comprising a suitable amount of each of the following:
a) snail secretion filtrate at a weight percentage of 1.5%;
b) copper tripeptide-1 at a weight percentage of 0.05%; and
c) sh-oligopeptide-1 at a weight percentage of 52%.

Most preferably, the present invention provides an anti-scarring composition comprising a suitable amount of ingredient at weight percentage recited in Table 3.

TABLE 3
ANTI-SCARRING COMPOSITION (MESO SCAR)

| RAW MATERIAL | | | |
|---|---|---|---|
| NAME | INCI NAME | FUNCTION | % |
| Water | Aqua | Solvent | 43.25% |
| HEC | Hydroxyethylcellulose | Thickener | 0.40% |
| Glycerin | Glycerin | Humectant | 1.00% |
| Propylene Glycol | Propylene Glycol | Humectant | 1.00% |
| Biosnail EZ | Snail Secretion Filtrate | Wound Healing, rapid skin regeneration | 1.50% |
| GHK-Cu | Copper Tripeptide-1 | Antiaging, Wound Healing, promote | 0.05% |
| | | epidermal growth, anti-wrinkle, | |
| | | wound hearling, increase skin | |
| | | elasticity & tenacity | |
| BIO-NanoEGF1 | sh-oligopeptide-1 | Anti-Aging, Hydration, Soothing, | 52.00% |
| | | skin rejuvenation, anti-aging, | |
| | | wrinkle prevention, wound healing, | |
| | | regeneration of the epidermis | |
| Nipaguard ® EHP | Phenoxyethanol (and) | Preservative | 0.80% |
| | Ethylhexylglycerin | | |

| | |
|---|---|
| TOTAL | 100.00% |

In some embodiments, the weight percentage of snail secretion filtrate is between about 0.5% to 2.5%.

In some embodiments, the weight percentage of snail secretion filtrate is between about 0.5% to 2%.

In some embodiments, the weight percentage of snail secretion filtrate is between about 1% to 2%.

In some embodiments, the weight percentage of snail secretion filtrate is 1.5%.

In some embodiments, the weight percentage of copper tripeptide-1 is between about 0.01% to 0.1%.

In some embodiments, the weight percentage of copper tripeptide-1 is between about 0.02% to 0.9%.

In some embodiments, the weight percentage of copper tripeptide-1 is between about 0.03% to 0.8%.

In some embodiments, the weight percentage of copper tripeptide-1 is between about 0.04% to 0.07%.

In some embodiments, the weight percentage of copper tripeptide-1 is between about 0.04% to 0.06%.

In some embodiments, the weight percentage of copper tripeptide-1 is 0.07%.

In some embodiments, the weight percentage of sh-oligopeptide-1 is between about 30-70%.

In some embodiments, the weight percentage of sh-oligopeptide-1 is between about 40-60%.

In some embodiments, the weight percentage of sh-oligopeptide-1 is between about 50-65%.

In some embodiments, the weight percentage of sh-oligopeptide-1 is between about 50-60%.

In some embodiments, the weight percentage of sh-oligopeptide-1 is between about 50-55%.

In some embodiments, the weight percentage of sh-oligopeptide-1 is 52%.

In some embodiments, the weight percentage of HEC is between about 0.5 to 0.6%.

In some embodiments, the weight percentage of HEC is 0.4%.

In some embodiments, the weight percentage of glycerin is between about 0.5 to 1.5%.

In some embodiments, the weight percentage of glycerin is 1%.

In some embodiments, the weight percentage of propylene glycol is between about 0.5-1.5%.

In some embodiments, the weight percentage of propylene glycol is 1%.

In some embodiments, the weight percentage of phenoxyethanol and ethylhexylglycerin is between about 0.6 to 1%.

In some embodiments, the weight percentage of phenoxyethanol and ethylhexylglycerin is 0.8%.

In some embodiments, the weight percentage of water is 35-45%.

In some embodiments, the weight percentage of water is 43.25%.

In some embodiments, the current invention provides a composition, wherein the weight percentage of
a) snail secretion filtrate is between about 1% to 2%;
b) copper tripeptide-1 is between about 0.04% to 0.06%; and
c) the weight percentage of sh-oligopeptide-1 is between about 50-55%.

In some embodiments, the current invention provides a composition of a formulation wherein the weight percentage of
a) snail secretion filtrate is 1.5%;
b) copper tripeptide-1 is 0.05%;
c) the weight percentage of sh-oligopeptide-1 is 52%;
d) HEC is 0.4%;
e) glycerin is 1%;
f) propylene glycol is 1%;
g) phenoxyethanol and ethylhexylglycerin is 0.8%; and
h) water is 43.25.

### Firming Composition

The present invention provides a firming composition comprising a suitable amount of each of the following:
a) water,
b) Glycerin,
c) Niacinamide,
d) Butylene Glycol,
e) 1,2-Hexanediol,
f) Caprylyl Glycol,
g) Lecithin,
h) Dipeptide Diaminobutyroyl Benzylamide Diacetate,
i) Acetyl Glutamine,
j) Sh-Oligopeptide-1,
k) Sh-Oligopeptide-2,
l) Sh-Polypeptide-1,
m) Sh-Polypeptide-9,
n) Sh-Polypeptide-11,
o) Bacillus/Folic Acid/Soybean Ferment Extract,
p) Sodium Hyaluronate,
q) Glutathione,
r) Hydroxyethylcellulose,
s) Disodium EDTA,
t) Phenoxyethanol, and
u) Ethylhexylglycerin.

Preferably, the present invention provides a firming composition comprising a suitable amount of each of the following:
a) niacinamide;
b) water, lecithin acetyl glutamine, sh-Oligopeptide-1, sh-Oligopeptide-2, sh-Polypeptide-1, sh-Polypeptide-9, sh-Polypeptide-11, Bacillus/Folic Acid/Soybean Ferment Extract, sodium hyaluronate, caprylyl glycol, butylene glycol and 1,2-Hexanediol;
c) glycerin, water, dipeptide diaminobutyroyl benzylamide diacetate;
d) sodium hyaluronate;
e) glutathione.

More preferably, the present invention provides a firming composition of a formulation comprising a suitable amount of each of the following:
a) niacinamide at a weight percentage of 4.5%;
b) water, lecithin acetyl glutamine, sh-Oligopeptide-1, sh-Oligopeptide-2, sh-Polypeptide-1, sh-Polypeptide-9, sh-Polypeptide-11, Bacillus/Folic Acid/Soybean Ferment Extract, sodium hyaluronate, caprylyl glycol, butvlene glycol and 1,2-Hexanediol a weight percentage of 3%;
c) glycerin, water, dipeptide diaminobutyroyl benzylamide diacetate a weight percentage of 4%;
d) sodium hyaluronate a weight percentage of 0.5%; and
e) glutathione a weight percentage of 0.5%.

Most preferably, the present invention provides an anti-scarring composition of a formulation comprising a suitable amount of ingredient at weight percentage recited in Table 4.

TABLE 4
FIRMING COMPOSITION (MESO FIRMING)

| RAW MATERIAL | | | |
|---|---|---|---|
| NAME | INCI NAME | FUNCTION | % |
| Water | Aqua | Solvent | 85.70% |
| HEC | Hydroxyethylcellulose | Thickener | 0.40% |
| Glycerin | Glycerin | Humectant | 1.00% |
| EDTA | Disodium EDTA | Chelating Agent | 0.10% |
| Niacinamide | Niacinamide | whitening, bluelight protection, anti- | 4.00% |
| | | inflammatory, skin brightening, anti-aging | |
| Hyamoist ® Care | Sodium Hyaluronate | Hydrating, Moisturizing, increase | 0.50% |
| Mini | | skin elasticity and hydration | |
| | | (sodium hyaluronate) | |
| Glutathione | Glutathione | Whitening, antioxidant, skin brightening | 0.50% |
| Bioplacenta | Water (and) Lecithin (and) Acetyl | Anti-Aging, Hydration, Soothing, | 3.00% |
| | Glutamine (and) sh-Oligopeptide-1 | anti-wrinkle, skin rejuvenation, | |
| | (and) sh-Oligopeptide-2 (and) sh- | elasticity enhancement, moisturizing, | |
| | Polypeptide-1 (and) sh-Polypeptide-9 | cell revitalization (max dose 3%). | |
| | (and) sh-Polypeptide-11 (and) | It is a combination of five | |
| | Bacillus/Folic Acid/Soybean Ferment | synthesized growth factors (EGF, | |
| | Extract (and) Sodium Hyaluronate | IGF-1, Acidic FGF, Basic FGF, VEGF), | |
| | (and) Caprylyl Glycol (and) Butylene | Vitamin B9, and Acetylated Glutamine | |
| | Glycol (and) 1,2-Hexanediol | | |
| Syn ®-AKE | Glycerin (and) Aqua (and) Dipeptide | Anti-aging, synthetic peptide that | 4.00% |
| | Diaminobutyroy1 Benzylamide | helps reduce the appearance of | |
| | Diacetate | wrinkles and laughter lines - fast | |
| | | (botox-like effect) | |
| Nipaguard ® | Phenoxyethanol (and) | Preservative | 0.80% |
| EHP | Ethylhexylglycerin | | |

| | |
|---|---|
| TOTAL | 100.00% |

In some embodiments, the weight percentage of niacinamide is between about 1% to 8%.

In some embodiments, the weight percentage of niacinamide is between about 2% to 7%.

In some embodiments, the weight percentage of niacinamide is between about 3% to 6%.

In some embodiments, the weight percentage of niacinamide is between about 3% to 5%.

In some embodiments, the weight percentage of niacinamide is 4%.

In some embodiments, the weight percentage of water, lecithin acetyl glutamine, sh-Oligopeptide-1, sh-Oligopeptide-2, sh-Polypeptide-1, sh-Polypeptide-9, sh-Polypeptide-11, Bacillus/Folic Acid/Soybean Ferment Extract, sodium hyaluronatc, caprylyl glycol, butylene glycol and 1,2-Hexanediol is between about 1% to 10%.

In some embodiments, the weight percentage of water, lecithin acetyl glutamine, sh-Oligopeptide-1, sh-Oligopeptide-2, sh-Polypeptide-1, sh-Polypeptide-9, sh-Polypeptide-11, Bacillus/Folic Acid/Soybean Ferment Extract. sodium hyaluronate, caprylyl glycol, butylene glycol and 1,2-Hexanediol is between about 2% to 9%.

In some embodiments, the weight percentage of water, lecithin acetyl glutamine, sh-Oligopeptide-1, sh-Oligopeptide-2, sh-Polypeptide-1, sh-Polvpeptide-9, sh-Polypeptide-11, Bacillus/Folic Acid/Soybean Ferment Extract. sodium hyaluronatc, caprylyl glycol, butylene glycol and 1,2-Hexanediol is between about 2% to 8%.

In some embodiments, the weight percentage of water, lecithin acetyl glutamine, sh-Oligopeptide-1, sh-Oligopeptide-2, sh-Polypeptide-1, sh-Polypeptide-9, sh-Polypeptide-11, Bacillus/Folic Acid/Soybean Ferment Extract. sodium hyaluronate, caprylyl glycol, butylene glycol and 1,2-Hexanediol is between about 2% to 7%.

In some embodiments, the weight percentage of water, lecithin acetyl glutamine, sh-Oligopeptide-1, sh-Oligopeptide-2, sh-Polypeptide-1, sh-Polvpeptide-9, sh-Polypeptide-11, Bacillus/Folic Acid/Soybean Ferment Extract. sodium hyaluronate, caprylyl glycol, butylene glycol and 1,2-Hexanediol is between about 2% to 6%.

In some embodiments, the weight percentage of water, lecithin acetyl glutamine, sh-Oligopeptidc-1, sh-Oligopeptide-2, sh-Polypeptide-1, sh-Polypeptide-9, sh-Polypeptide-11, Bacillus/Folic Acid/Soybean Ferment Extract. sodium hyaluronate, caprylyl glycol, butylene glycol and 1,2-Hexanediol is between about 2% to 5%.

In some embodiments, the weight percentage of water, lecithin acetyl glutamine, sh-Oligopeptide-1, sh-Oligopeptide-2, sh-Polypeptide-1, sh-Polypeptide-9, sh-Polypeptide-11, Bacillus/Folic Acid/Soybean Ferment Extract. sodium hyaluronate, caprylyl glycol, butylene glycol and 1,2-Hexanediol is between about 2% to 4%.

In some embodiments, the weight percentage of water, lecithin acetyl glutamine, sh-Oligopeptide-1, sh-Oligopeptide-2, sh-Polypeptide-1, sh-Polypeptide-9, sh-Polypeptide-11, Bacillus/Folic Acid/Soybean Ferment Extract. sodium hyaluronate, caprylyl glycol, butylene glycol and 1,2-Hexanediol is 3%.

In some embodiments, the weight percentage of glycerin, water, dipeptide diaminobutyroyl benzvlamide diacetate is between about 1% to 8%.

In some embodiments, the weight percentage of glycerin, water, dipeptide diaminobutyroyl benzvlamide diacetate is between about 2% to 7%.

In some embodiments, the weight percentage of glycerin, water, dipeptide diaminobutyroyl benzylamide diacetate is between about 3% to 6%.

In some embodiments, the weight percentage of glycerin, water, dipeptide diaminobutyroyl benzylamide diacetate is between about 3% to 5%.

In some embodiments, the weight percentage of glycerin, water, dipeptidc diaminobutyroyl benzylamide diacetate is 4%.

In some embodiments, the weight percentage of sodium hyaluronate is between about 0.1-0.8%.

In some embodiments, the weight percentage of sodium hyaluronate is between about 0.2-0.7%.

In some embodiments, the weight percentage of sodium hyaluronate is between about 0.3-0.6%.

In some embodiments, the weight percentage of sodium hyaluronate is 0.5%.

In some embodiments, the weight percentage of glutathione is between about 0.1-0.8%.

In some embodiments, the weight percentage of glutathione is between about 0.2-0.7%.

In some embodiments, the weight percentage of glutathione is between about 0.3-0.6%.

In some embodiments, the weight percentage of glutathione is 0.5%.

In some embodiments, the weight percentage of HEC is between about 0.2-0.6%.

In some embodiments, the weight percentage of HEC is 0.4%.

In some embodiments, the weight percentage of glycerin is between about 0.5-1.5%.

In some embodiments, the weight percentage of glycerin is 1%.

In some embodiments, the weight percentage of disodium EDTA is between about 0.05-0.15%.

In some embodiments, the weight percentage of disodium EDTA is 0.1%.

In some embodiments, the weight percentage of phenoxyethanol and ethylhexylglycerin is between about 0.6-1.0%.

In some embodiments, the weight percentage of phenoxyethanol and ethylhexylglycerin is 0.8%.

In some embodiments, the weight percentage of water is 80-90%

In some embodiments, the weight percentage of water is 85.7%.

In some embodiments, the present invention provides a composition of a formulation wherein
a) niacinamide is between about 3% to 5%;
b) water, lecithin acetyl glutamine, sh-Oligopeptide-1, sh-Oligopeptide-2, sh-Polypeptide-1, sh-Polypeptide-9, sh-Polypeptide-11, Bacillus/Folic Acid/Soybean Ferment Extract. sodium hyaluronate, caprylyl glycol, butylene glycol and 1,2-Hexanediol is between about 2% to 4%;
c) sodium hyaluronate is between about 0.3-0.6%;
d) glutathione is between about 0.3-0.6%; and
e) glycerin, water, dipeptide diaminobutyroyl benzylamide diacetate is between about 3% to 5%.

In some embodiments, the present invention provides a composition of a formulation wherein
a) niacinamide is 4%;
b) water, lecithin acetyl glutamine, sh-Oligopeptide-1, sh-Oligopeptide-2, sh-Polypeptide-1, sh-Polypeptide-9, sh-Polypeptide-11, Bacillus/Folic Acid/Soybean Ferment Extract. sodium hyaluronate, caprylyl glycol, butylene glycol and 1,2-Hexanediol is 3%;
c) glycerin, water, dipeptide diaminobutyroyl benzylamide diacetate is 4%;
d) sodium hyaluronate is 0.5%;
e) glutathione is 0.5%;
f) HEC is 0.4%;
g) glycerin is 1%;
h) disodium EDTA is 0.1%; and
i) phenoxyethanol and ethylhexylglycerin is 0.8%.

### Anti-Aging Composition

The present invention provides an anti-aging composition comprising a suitable amount of each of the following:
a) water,
b) niacinamide,
c) propylene glycol,
d) glycerin,
e) butylene glycol,
f) tranexamic acid,
g) rumex occidentalis extract,
h) ascorbic acid,
i) acetyl tyrosine,
j) aminopropyl ascorbyl phosphate,
k) sxifraga sarmentosa extract,
l) arbutin,
m) paeonia suffruticosa extract,
n) scutellaria baicalensis root extract,
o) glutathione,
p) hydroxyethylcellulose (HEC),
q) citric acid,
r) sodium sulfite,
s) disodium EDTA,
t) phenoxyethanol, and
u) ethylhexylglycerin.

Preferably, the present invention provides an anti-aging composition comprising a suitable amount of each of the following:
a) water, butylene glycol, citric acid, acetyl tyrosine, sodium sulfite, aminopropyl ascorbyl phosphate, saxifraga sarmentosa extract, arbutin. paeonia suffructicosa extract, scutellaria baicalensis, root extract, and glutathione is between about 2-4%. water, lecithin acetyl glutamine, sh-Oligopeptide-1, sh-Oligopeptide-2, sh-Polypeptide-1, sh-Polypeptide-9, sh-Polypeptide-11, Bacillus/Folic Acid/Soybean Ferment Extract. sodium hyaluronate, caprylyl glycol, butylene glycol and 1,2-Hexanediol;
b) niacinamide;
c) water, glycerin, rumex occidentalis extract, ascorbic acid; and
d) tranexamic acid.

More preferably, the present invention provides an anti-aging composition comprising a suitable amount of each of the following:
a) water, butylene glycol, citric acid, acetyl tyrosine, sodium sulfite, aminopropyl ascorbyl phosphate, saxifraga sarmentosa extract, arbutin, paeonia suffructicosa extract, scutellaria baicalensis, root extract, and glutathione is between about 2-4% water, lecithin acetyl glutamine, sh-Oligopeptide-1, sh-Oligopeptide-2, sh-Polypeptide-1, sh-Polypeptide-9, sh-Polypeptide-11, Bacillus/Folic Acid/Soybean Ferment Extract. sodium hyaluronate, caprylyl glycol, butylene glycol and 1,2-Hexanediol is at aweight percentage of 3%;
b) niacinamide is at aweight percentage of 4%,
c) water, glycerin, rumex occidentalis extract, ascorbic acid is at a weight percentage of 2%; and
d) tranexamic acid is at a weight percentage of 1%.

Most preferably, the present invention provides an anti-aging composition comprising a suitable amount of ingredient at weight percentage recited in Table 5.

TABLE 5
ANTI-AGING COMPOSITION (MESO DARKSPOT)

| RAW MATERIAL | | | |
|---|---|---|---|
| NAME | INCI NAME | FUNCTION | % |
| Water | Aqua | Solvent | 86.70% |
| HEC | Hydroxyethylcellulose | Thickener | 0.40% |
| Glycerin | Glycerin | Humectant | 1.00% |
| Propylene Glycol | Propylene glycol | Humectant | 1.00% |
| EDTA | Disodium EDTA | Chelating Agent | 0.10% |
| Clair Blanche II | Water (and) Butylene Glycol (and) Citric | Brightening, Antixoxidant, | 3.00% |
| | Acid (and) Acetyl tyrosine (and) Sodium | multifunctional and complex | |
| | Sulfite (and) Aminopropyl Ascorbyl | whitening ingredient targeting | |
| | Phosphate (and) Saxifraga Sarmentosa | pigmentation, antioxidant, anti- | |
| | Extract (and) Arbutin (and) Paeonia | inflammatory | |
| | Suffruticosa Extract (and) Scutellaria | | |
| | Baicalensis Root Extract (and) Glutathione | | |
| Niacinamide | Niacinamide | Anti-Aging, Pore Minimizer, | 4.00% |
| | | Soothing, anti-inflammatory, | |
| | | skin brightening, anti-aging | |
| Tyrostat ™ - 11 | Water (and) Glycerin (and) Rumex | Brightening, Antioxidant, | 2.00% |
| | Occidentalis Extract (and) Ascorbic Acid | Tyrostat ™ safely reduces skin | |
| | | pigmentation and erythema by | |
| | | limiting both melanin | |
| | | production and skin reddening | |
| | | for an even skin complexion. It | |
| | | has higher activity than arbutin, | |
| | | turmeric and hydroquinone with | |
| | | no unwanted side effects | |
| Tranexamic Acid | Tranexamic Acid | Brightening, (Tranexamic Acid) | 1.00% |
| | | potent skin whitening, anti- | |
| | | inflammatory | |
| Nipaguard ® EHP | Phenoxyethanol (and) Ethylhexylglycerin | Preservative | 0.80% |

| | |
|---|---|
| TOTAL | 100.00% |

In some embodiments, the weight percentage of water, butylene glycol, citric acid, acetyl tyrosine, sodium sulfite, aminopropyl ascorbyl phosphate, saxifraga sarmentosa extract, arbutin, paconia suffructicosa extract, scutellaria baicalensis, root extract, and glutathione is between about 1-8%.

In some embodiments, the weight percentage of water, butylene glycol, citric acid, acetyl tyrosine, sodium sulfite, aminopropyl ascorbyl phosphate, saxifraga sarmentosa extract, arbutin, paeonia suffructicosa extract, scutellaria baicalensis, root extract, and glutathione is between about 1-7%.

In some embodiments, the weight percentage of water, butylene glycol, citric acid, acetyl tyrosine, sodium sulfite, aminopropyl ascorbyl phosphate, saxifraga sarmentosa extract, arbutin, paeonia suffructicosa extract, scutellaria baicalensis, root extract, and glutathione is between about 1-6%.

In some embodiments, the weight percentage of water, butylene glycol, citric acid, acetyl tyrosine, sodium sulfite, aminopropyl ascorbyl phosphate, saxifraga sarmentosa extract, arbutin, paeonia suffructicosa extract, scutellaria baicalensis, root extract, and glutathione is between about 2-5%.

In some embodiments, the weight percentage of water, butylene glycol, citric acid, acetyl tyrosine, sodium sulfite, aminopropyl ascorbyl phosphate, saxifraga sarmentosa extract, arbutin, paeonia suffructicosa extract, scutellaria baicalensis, root extract, and glutathione is between about 2-4%.

In some embodiments, the weight percentage of water, butylene glycol, citric acid, acetyl tyrosine, sodium sulfite, aminopropyl ascorbyl phosphate, saxifraga sarmentosa extract, arbutin, paeonia suffructicosa extract, scutellaria baicalensis, root extract, and glutathione is 3%.

In some embodiments, the weight percentage of niacinamide is between about 1-9%.

In some embodiments, the weight percentage of niacinamide is between about 1-8%.

In some embodiments, the weight percentage of niacinamide is between about 1-7%.

In some embodiments, the weight percentage of niacinamide is between about 2-6%.

In some embodiments, the weight percentage of niacinamide is between about 3-5%.

In some embodiments, the weight percentage of niacinamide is about 4%.

In some embodiments, the weight percentage of water, glycerin, rumex occidentalis extract, ascorbic acid is between about 0.5-5%.

In some embodiments, the weight percentage of water, glycerin, rumex occidentalis extract, ascorbic acid is between about 0.5-4.5%.

In some embodiments, the weight percentage of water, glycerin, rumex occidentalis extract, ascorbic acid is between about 0.54%.

In some embodiments, the weight percentage of water, glycerin, rumex occidentalis extract, ascorbic acid is between about 0.5-3.5%.

In some embodiments, the weight percentage of water, glycerin, rumex occidentalis extract, ascorbic acid is between about 1-3%.

In some embodiments, the weight percentage of water, glycerin, rumex occidentalis extract, ascorbic acid is 2%.

In some embodiments, the weight percentage oftranexamic acid is between about 0.2-1.8%.

In some embodiments, the weight percentage oftranexamic acid is between about 0.4-1.6%.

In some embodiments, the weight percentage oftranexamic acid is between about 0.6-1.4%.

In some embodiments, the weight percentage of tranexamic acid is between about 0.8-1.2%.

In some embodiments, the weight percentage of tranexamic acid is 1%.

In some embodiments, the weight percentage of HEC is between about 0.2-0.6%.

In some embodiments, the weight percentage of HEC is 0.4%.

In some embodiments, the weight percentage of glycerin is between about 0.5-1.5%.

In some embodiments, the weight percentage of glycerin is 1%.

In some embodiments, the weight percentage of disodium EDTA is between about 0.05-0.15%.

In some embodiments, the weight percentage of disodium EDTA is 0.1%.

In some embodiments, the weight percentage of phenoxyethanol and ethylhexylglycerin is between about 0.6-1.0%.

In some embodiments, the weight percentage of phenoxyethanol and ethylhexylglycerin is 0.8%.

In some embodiments, the weight percentage of propylene glycol is between about 0.5-1.5%.

In some embodiments, the weight percentage of propylene glycol is 1%.

In some embodiments, the weight percentage of water is 35-45%.

In some embodiments, the weight percentage of water is 43.25%.

In some embodiments, the present invention provides a composition of a formulation wherein the weight percentage of
a) water, butylene glycol, citric acid, acetyl tyrosine, sodium sulfite, aminopropyl ascorbyl phosphate, saxifraga sarmentosa extract, arbutin, paeonia suffructicosa extract, scutellaria baicalensis, root extract, and glutathione is between about 2-4% water, lecithin acetyl glutamine, sh-Oligopeptide-1, sh-Oligopeptide-2, sh-Polypeptide-1, sh-Polypeptide-9, sh-Polypeptide-11, Bacillus/Folic Acid/Soybean Ferment Extract. sodium hyaluronate, caprylyl glycol, butylene glycol and 1,2-Hexanediol is between about 2% to 4%;
b) niacinamide is between about 3-5%;
c) water, glycerin, rumex occidentalis extract, ascorbic acid is between about 1-3%; and
d) tranexamic acid is between about 0.8-1.2%.

In some embodiments, the present invention provides a composition of a formulation wherein the weight percentage of
a) water, butylene glycol, citric acid, acetyl tyrosine, sodium sulfite, aminopropyl ascorbyl phosphate, saxifraga sarmentosa extract, arbutin, paeonia suffructicosa extract, scutellaria baicalensis, root extract, and glutathione is between about 2-4% water, lecithin acetyl glutamine, sh-Oligopeptide-1, sh-Oligopeptide-2, sh-Polypeptide-1, sh-Polypeptide-9, sh-Polypeptide-11, Bacillus/Folic Acid/Soybean Ferment Extract. sodium hyaluronate, caprylyl glycol, butylene glycol and 1,2-Hexanediol is 3%;
b) niacinamide is 4%;
c) water, glycerin, rumex occidentalis extract, ascorbic acid is 2%; and
d) tranexamic acid is between about 1%.

A composition of a formulation comprising a mixture of any suitable amount of the compositions stated above.

In some embodiments, the composition is in the form of serum, ointment, gel, or cream.

The invention also includes 40-60% refining composition by volume, with the remainder being the brightening composition, preferably, 50% refining composition and 50% brightening composition by volume.

The invention also includes 40-60% anti-scaring composition by volume, with the remainder being the brightening composition, preferably, 50% anti-scaring composition and 50% brightening composition by volume.

The invention also includes 40-60% firming composition by volume, with the remainder being the anti-aging composition, preferably, 50% firming composition and 50% anti-aging composition by volume.

In some embodiments, the composition has a volume of about 0.1-1 cubic centimeter (cc).

In some embodiments, the composition has a volume of about 0.2-0.9 cc.

In some embodiments, the composition has a volume of about 0.3-0.8 cc.

In some embodiments, the composition has a volume of about 0.4-0.6 cc.

In some embodiments, the composition has a volume of 0.5 cc.

The present invention provides a method of increasing skin brightness of an individual, wherein the method comprises applying the individual's skin an effective amount of a brightening composition and refining composition, or ani-scaring composition and brightening composition, or firming composition and anti-aging composition.

The present invention provides a method of increasing skin firmness of an individual, wherein the method comprises applying the individual's skin an effective amount of a brightening composition and refining composition, or ani-scaring composition and brightening composition, or firming composition and anti-aging composition.

The present invention provides a method of increasing skin smoothness of an individual, wherein the method comprises applying the individual's skin an effective amount of a brightening composition and refining composition, or ani-scaring composition and brightening composition, or firming composition and anti-aging composition.

The present invention provides a method of decreasing in fine lines/wrinkle of an individual, wherein the method comprises applying the individual's skin an effective amount of a brightening composition and refining composition, or ani-scaring composition and brightening composition, or firming composition and anti-aging composition.

The present invention provides a method of fading melasma/hyperpigmentation of an individual, wherein the method comprises applying the individual's skin an effective amount of a brightening composition and refining composition, or ani-scaring composition and brightening composition, or firming composition and anti-aging composition.

The present invention provides a method of fading/shallowing facial marks of an individual, wherein the method comprises applying the individual's skin an effective amount of a brightening composition and refining composition, or ani-scaring composition and brightening composition, or firming composition and anti-aging composition.

In some embodiments of the method, the composition is applied invasively.

In some embodiments of the method, the composition is applied non-invasively.

In some embodiments of the method, the composition is applied while microneedling.

In some embodiments of the method, the facial marks comprise moles, freckles, pimples, scars, acne marks.

In some embodiments of the method, the facial marks are acne marks.

In some embodiments of the method, the composition is applied for at least 1, 2, 3, 4, 5, or 6 sessions.

In some embodiments of the method, the composition is applied for at least 2, 3, 4, 5, or 6 sessions.

In some embodiments of the method, the composition is applied for at least 3, 4, 5, or 6 sessions.

In some embodiments of the method, the composition is applied for at least 4, 5, or 6 sessions.

In some embodiments of the method, the composition is applied for at least 3 sessions.

In some embodiments of the method, the composition is applied for at least 5 or 6 sessions.

In some embodiments of the method, the time period between each session is the same.

In some embodiments of the method, the time period between each session is different.

In some embodiments of the method, the period between each session is at least 3, 4, 5, 6, 7, 8, 9, or 10 days.

In some embodiments of the method, the period between each session is at least 5 6, 7, 8 or 9 days.

In some embodiments of the method, the period between each session is at least 5 6, 7, 8 or 9 days.

In some embodiments of the method, the period between each session is at least 7 days.

In some embodiments of the method, the period between each session is 7 days.

In some embodiments of the method, the composition is applied using a Dermaroller and/or a Dermapen.

In some embodiments of the method, wherein the composition applied in the first session is applied with a Dermaroller.

In some embodiments of the method, the Dermaroller has needles, wherein the length of the needles is 1-3 mm.

In some embodiments of the method, the Dermaroller has needles, wherein the length of the needles is 1.5-2.5 mm.

In some embodiments of the method, the Dermaroller has needles, wherein the length of the needles is 1.5 or 2 mm.

In some embodiments of the method, the composition applied in the second session is applied with a Dermapen.

In some embodiments of the method, the Dermapen has needles, where in the length of the needles is 1-3 mm.

In some embodiments of the method, the Dermapen has needles, where in the length of the needles is 1.5-2.5 mm.

In some embodiments of the method, the Dermapen has needles, where in the length of the needles is 1.5 or 2 mm.

In some embodiments of the method, further comprising apply a numbing cream and/or a topical anesthesia cream.

In some embodiments of the method, further comprising apply a numbing cream and topical anesthesia cream at least 30-60 mins prior to each session.

In some embodiments of the method, further comprising apply a numbing cream and topical anesthesia cream at least 45 mins prior to each session.

In some embodiment, the present invention provides an article containing the composition stated above.

The present invention provides a formulation comprising a component A and a component B, wherein the component A comprises:
a) niacinamide;
b) sodium hyaluronate;
c) 1,2-Hexanediol;
d) Sh-Oligopeptide-1;
e) Sh-Oligopeptide-2;
f) Sh-Polypeptide-1;
g) Sh-Polypeptide-9;
h) Sh-Polypeptide-11;
i) dipeptide diaminobutyroyl benzylamide diacetate; and
j) bacillus, folic acid, and/or soybean ferment extract; and
   wherein the component B comprises:
a) niacinamide;
b) tranexamic acid;
c) rumex occidentalis extract;
d) ascorbic acid;
e) aminopropyl ascorbyl phosphate;
f) sxifraga sarmentosa extract;
g) arbutin;
h) paeonia suffruticosa extract;
i) scutellaria baicalensis root extract; and
j) citric acid.

In some embodiments, wherein the component A and B, each individually, further comprises one or more of a solvent, a chelating agent, an antioxidant agent, a preservative, a skin-conditioning agent, a humectant, a thickening agent, and a dispersing agent.

In some embodiments, the solvent is selected from a list consisting of water, ethanol (ethyl alcohol), isopropyl alcohol, propylene glycol, butylene glycol, glycerin, dimethyl isosorbide, caprylic/capric triglyceride, cyclopentasiloxane, dimethicone, mineral oil, silicones (e.g., cyclomethicone, dimethicone copolyol), glycols (e.g., hexylene glycol, diethylene glycol), polyethylene glycol (peg), butyl acetate, ethyl acetate, acetone, isododecane, squalane, and triethyl citrate.

In some embodiments, the chelating agent is selected from a list consisting of edetate disodium (disodium EDTA), EDTA, citric acid, sodium citrate, sodium gluconate, phytic acid, gluconolactone, hydroxyethyl ethylenediamine triacetic acid (HEDTA), tetrasodium glutamate diacetate, trisodium ethylenediamine disuccinate (Trisodium EDTA), sodium phytate, potassium citrate, sodium lactate, sodium malate, sodium tartrate, and sodium ascorbate.

In some embodiments, the antioxidant agent is selected from a list consisting of glutathione, tocopherol, ascorbic acid, retinol, ubiquinone, niacinamide, resveratrol, ferulic acid, alpha lipoic acid, green tea extract, grape seed extract, coenzyme Q10, astaxanthin, pomegranate extract, rosemary extract, vitamin C, vitamin E, idebenone, lycopene, beta-carotene, and tea tree oil.

In some embodiments, preservative is selected from a list consisting of phenoxyethanol, sodium sulfite, ethylhexylglycerin, parabens, phenoxyethanol, benzyl alcohol, chlorphenesin, ethylhexylglycerin, potassium sorbate, sodium benzoate, caprylyl glycol, hexylene glycol, benzoic acid, sorbic acid, sodium hydroxymethylglycinate, dehydroacetic acid, salicylic acid, ethylparaben, butylparaben, triclosan, iodopropynyl butylcarbamate (IPBC), and phthalates, formaldehyde-releasing agents.

In some embodiments, the skin-conditioning agent is selected from a list consisting of acetyl glutamine, acetyl tyrosine, propylene glycol, butylene glycol, glycerin, hyaluronic acid, shea butter, cocoa butter, aloe vera, squalane, jojoba oil, almond oil, coconut oil, olive oil, panthenol, niacinamide, ceramides, collagen, elastin, peptides, allantoin, honey, vitamin E, and argan oil.

In some embodiments, the humectant is selected from a list consisting of caprylyl glycol, glycerin, hyaluronic acid, sorbitol, propylene glycol, butylene glycol, urea, alpha hydroxy acids (AHAs), honey, aloe vera, sodium lactate, sodium PCA, panthenol, and allantoin.

In some embodiments, the thickening agent is selected from a list consisting of hydroxyethylcellulose, carbomer, xanthan gum, cellulose gum, guar gum, acrylates/C10-30 alkyl acrylate crosspolymer, carrageenan, silica, bentonite, kaolin, starch, magnesium aluminum silicate, and hectorite.

In some embodiments, the dispersing agent is selected from a list consisting of lecithin, propylene glycol, glycerin, sorbitan oleate, polysorbate 20, polysorbate 80, peg-40 hydrogenated castor oil, ceteareth-20, cetearyl alcohol, steareth-20, and ethoxylated alcohols.

In some embodiments,
a) the solvent is water and/or butylene glycol;
b) the chelating agent is edetate disodium (disodium EDTA);
c) the antioxidant agent is glutathione;
d) the preservative is phenoxyethanol, sodium sulfite, and/or ethylhexylglycerin;
e) the skin-conditioning agent is acetyl glutamine, propylene glycol, and/or acetyl tyrosine;
f) the humectant is caprylyl glycol and/or glycerin;
g) the thickening agent is hydroxyethyl cellulose; and/or
h) the dispersing agent is lecithin.

In some embodiments, the weight percentage of
a) niacinamide is about 3%-6%;
b) water, lecithin acetyl glutamine, sh-oligopeptide-1, sh-oligopeptide-2, sh-polypeptide-1, sh-polypeptide-9, sh-polypeptide-11, bacillus, folic acid, and/or soybean ferment extract, sodium hyaluronate, caprylyl glycol, butylene glycol and 1,2-hexanediol are about 2% to 5%;
c) glycerin, water, and dipeptide diaminobutyroyl benzylamide diacetate are about 2% to 7%;
d) sodium hyaluronate is about 0.2-0.7%;
e) glutathione is about 0.2-0.7%;
f) HEC is about 0.2-0.6%;
g) glycerin is about 0.5-1.5%;
h) disodium EDTA is about 0.05-0.15%;
i) phenoxyethanol and ethylhexylglycerin are between 0.6-1.0%; and/or
j) water is about 80-90%.

In some embodiments, the weight percentage of
a) niacinamide is about 3/6-5%;
b) water, lecithin acetyl glutamine, sh-oligopeptide-1, sh-oligopeptide-2, sh-polypeptide-1, sh-polypeptide-9, sh-polypeptide-11, bacillus, folic acid, and/or soybean ferment extract, sodium hyaluronate, caprylyl glycol, butylene glycol and 1,2-hexanediol are about 2%-4%;
c) glycerin, water, and dipeptide diaminobutyroyl benzylamide diacetate are between about 3% to 5%;
d) sodium hyaluronate is about 0.2-0.7%; and/or
e) glutathione is about 0.3-0.6%.

In some embodiments, the weight percentage of
a) niacinamide is about 3/6-5%;
b) water, lecithin, acetyl glutamine, sh-oligopeptide-1, sh-oligopeptide-2, sh-polypeptide-1, sh-polypeptide-9, sh-polypeptide-11, bacillus, folic acid, and/or soybean ferment extract, sodium hyaluronate, caprylyl glycol, butylene glycol and 1,2-hexanediol are about 2%-4%; c) sodium hyaluronate is about 0.3-0.6%;
d) glutathione is about 0.3-0.6%; and
e) glycerin, water, and dipeptide diaminobutyroyl benzylamide diacetate are about 3% to 5%.

In some embodiments, the weight percentage of
a) niacinamide is 4%;
b) water, lecithin acetyl glutamine, sh-Oligopeptide-1, sh-oligopeptide-2, sh-polypeptide-1, sh-polypeptide-9, sh-polypeptide-11, bacillus, folic acid, and/or soybean ferment extract, sodium hyaluronate, caprylyl glycol, butylene glycol and 1,2-hexanediol are 3%;
c) glycerin, water, and dipeptide diaminobutyroyl benzylamide diacetate are 4%;
d) sodium hyaluronate is 0.5%;
e) glutathione is 0.5%;
f) HEC is 0.4%;
g) glycerin is 1%;
h) disodium EDTA is 0.1%; and
i) phenoxyethanol and ethylhexylglycerin are 0.8%.

In some embodiments, the weight percentage of
a) water, butylene glycol, citric acid, acetyl tyrosine, sodium sulfite, aminopropyl ascorbyl phosphate, saxifraga sarmentosa extract, arbutin, paconia suffructicosa extract, scutellaria baicalensis, root extract, and glutathione are about 2-5%;
b) niacinamide is about 2-6%;
c) water, glycerin, rumex occidentalis extract, and ascorbic acid are about 0.5-3.5%; d) tranexamic acid is about 0.6-1.4%;
e) HEC is about 0.2-0.6%;
f) glycerin is about 0.5-1.5%;
g) disodium EDTA is about 0.05-0.15%;
h) phenoxyethanol and ethylhexylglycerin are about 0.6-1.0%;
i) propylene glycol is about 0.5-1.5%; and/or
j) water is 35-45%.

In some embodiments, the weight percentage of
a) water, butylene glycol, citric acid, acetyl tyrosine, sodium sulfite, aminopropyl ascorbyl phosphate, saxifraga sarmentosa extract, arbutin, paeonia suffructicosa extract, scutellaria baicalensis, root extract, and glutathione are about 2-5%;
b) niacinamide is about 2-6%;
c) water, glycerin, rumex occidentalis extract, and ascorbic acid are about 0.5-3.5%;
d) tranexamic acid is about 0.6-1.4%;
e) HEC is about 0.2-0.6%;
f) glycerin is about 0.5-1.5%;
g) disodium EDTA is about 0.05-0.15%;
h) phenoxyethanol and ethylhexylglycerin are about 0.6-1.0%;
i) propylene glycol is about 0.5-1.5%; and/or
j) water is 35-45%.

In some embodiments, the weight percentage of
a) water, butylene glycol, citric acid, acetyl tyrosine, sodium sulfite, aminopropyl ascorbyl phosphate, saxifraga sarmentosa extract, arbutin, paeonia suffructicosa extract, scutellaria baicalensis, root extract, and glutathione are about 2-4%;
b) niacinamide is about 3-5%;
c) water, glycerin, rumex occidentalis extract, and ascorbic acid is about 1-3%; and
d) tranexamic acid is about 0.8-1.2%.

In some embodiments, the weight percentage of
a) water, butylene glycol, citric acid, acetyl tyrosine, sodium sulfite, aminopropyl ascorbyl phosphate, saxifraga sarmentosa extract, arbutin, paeonia suffructicosa extract, scutellaria baicalensis, root extract, and glutathione are about 3%;
b) niacinamide is about 4%;
c) water, glycerin, rmex occidentalis extract, and ascorbic acid is about 2%;
d) tranexamic acid is about 1%;
e) HEC is about 0.4%;
f) glycerin is about 1%;
g) disodium EDTA is about 0.1%;
h) phenoxyethanol and ethylhexylglycerin are about 0.8%;
i) propylene glycol is about 1%; and/or
j) water is 43.25%.

The present invention provides a composition of a formulation comprising a component A and a component B,
wherein the component A comprises:
   a) sh-Oligopeptide-1;
   b) niacinamide;
   c) laminaria saccharina extract; and
   d) starfish coelomic fluid extract; and
wherein the component B comprises:
   a) kojic acid;
   b) glycolic acid;
   c) potassium azelaoyl diglycinate;
   d) sodium salt of pyroglutamic acid (sodium PCA);
   e) arctostaphylos uva-ursi leaf extract;
   f) magnesium ascorbyl phosphate;
   g) alpha-arbutin;
   h) sodium benzoate; and
   i) sodium metabisulfite.

The present invention provides composition of a formulation comprising a component A and a component B,
wherein the component A comprises:
   a) kojic acid;
   b) glycolic acid;
   c) potassium azelaoyl diglycinate;
   d) sodium salt of pyroglutamic acid (sodium PCA);
   e) arctostaphylos uva-ursi leaf extract;
   f) magnesium ascorbyl phosphate;
   g) alpha-arbutin;
   h) sodium benzoate; and
   i) sodium metabisulfite; and
wherein the component B comprises:
   a) sh-oligopeptide-1;
   b) snail secretion filtrate; and
   c) copper tripeptide-1.

Each embodiment disclosed herein is contemplated as being applicable to each of the other disclosed embodiments. Thus, all combinations of the various elements described herein are within the scope of the invention. In addition, the elements recited in the composition embodiments can be used in the method and use embodiments described herein and vice versa.

The present invention is illustrated and further described in more detail with reference to the following non-limiting examples. The following examples illustrate the practice of the present subject matter in some of its embodiments but should not be construed as limiting the scope of the present subject matter. Other embodiments will be apparent to one skilled in the art from consideration of the specification and examples. It is intended that the specification, including the examples, is considered exemplary only without limiting the scope and spirit of the present subject matter.

All combinations of the various elements disclosed herein are within the scope of the invention.

As used herein, all headings are simply for organization and are not intended to limit the disclosure in any manner. The content of any individual section may be equally applicable to all sections. All combinations of the various elements disclosed herein are within the scope of the invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context ofa single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

This invention will be better understood by reference to the Examples which follow, but those skilled in the art will readily appreciate that the specific experiments detailed are only illustrative of the invention as described more fully in the claims which follow thereafter.

### EXAMPLES

### Example 1

A dermatologist administered 0.5 cc refining composition as described in Table 1 and 0.5 cc brightening composition as described in Table 2 to patients. There are three sessions with 3 weeks interval per session.

5 Female and 1 Male Volunteers, 25-49 years old were participated in the study, which have skin type of Normal & Combination skin. Dull. Rough Skin, Skin discoloration were observed before applying the composition.

Dermaroller (1.5 mm) was used for the first session and Dermapen (1.5 mm) was used from the Second session onwards. A combination of numbing cream and topical anesthesia was applied at least 45 minutes prior to each session.

The primary objective of the study was to evaluate the efficacy of the treatment in improving skin texture, and brightness, and minimizing pores and fine lines. The secondary objective was to assess the safety and tolerability of the treatment on the participants. This study evaluated the outcomes of three sessions with a 3-week interval per session, administered by a dermatologist.

### Method

### Study Design:

The clinical trial was a prospective, single-arm study designed to assess the efficacy of the combined use of refining composition serum and brightening composition serum for improving skin texture, reducing fine lines, and addressing skin discoloration.

The study involved three sessions with 3-week intervals per session. Dermaroller and Dermapen were used for the first and subsequent sessions, respectively. Numbing cream and topical anesthesia were applied before each session.

### Study Participants

The study participants were 4 female and 1 male volunteer aged between 25-49 years old, who had normal and combination skin with dull, rough skin, and skin discoloration.

Intervention: The study intervention consisted of administering 0.5 cc refining composition serum and 0.5 cc brightening composition serum to the participants in each session, using Dermaroller for the first session (1.5 mm) and Dermapen from the second session onwards (1.5 mm). A combination of numbing cream and topical anesthesia was applied at least 45 minutes prior to each session.

### Outcome Measures

The primary outcome measures of the study were improvements in skin texture, reduction in fine lines, and addressing skin discoloration. These outcomes were assessed by evaluating the participants' skin condition after 9 weeks of the intervention. The secondary outcome measures were the safety and tolerability of the intervention.

### Data Analysis:

The data analysis was conducted by comparing the participants' skin conditions before and after the intervention using descriptive statistics. The primary outcome measures were evaluated by assessing changes in skin texture, fine lines, and skin discoloration. The safety and tolerability of the intervention were evaluated by assessing adverse events and participant feedback.

### Results

After nine weeks of treatment, all study participants reported significant improvements in their skin texture, with even skin tone, smaller pores, and fewer fine lines. The following results were observed during the study:

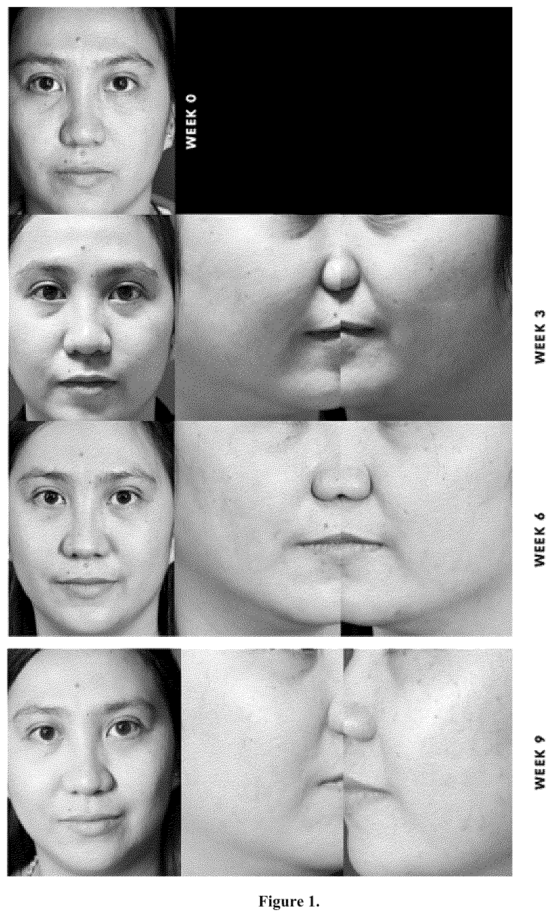

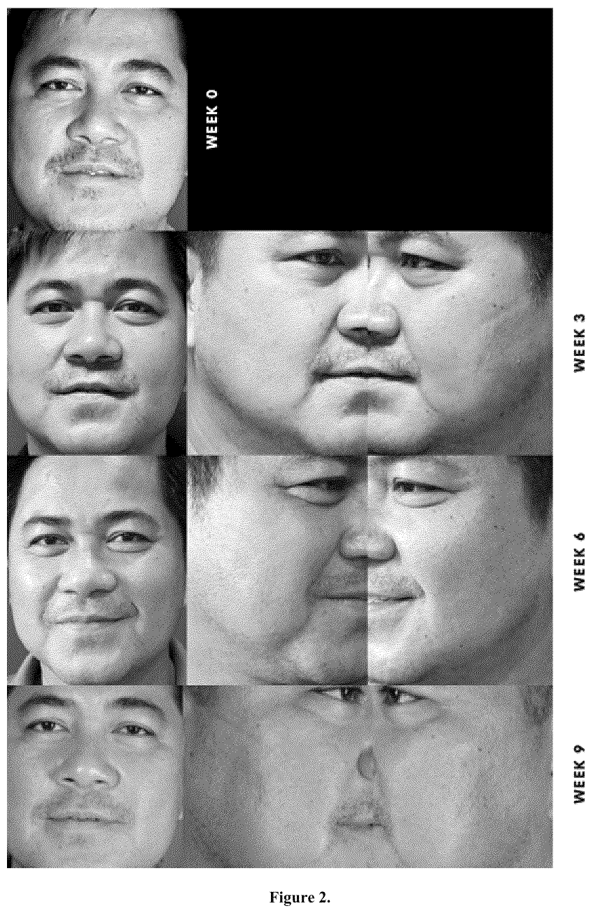

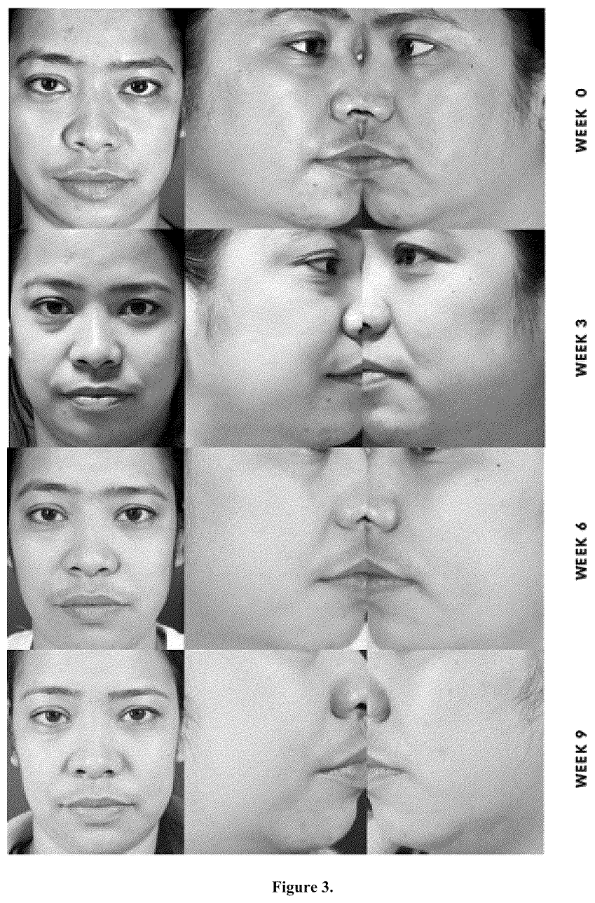

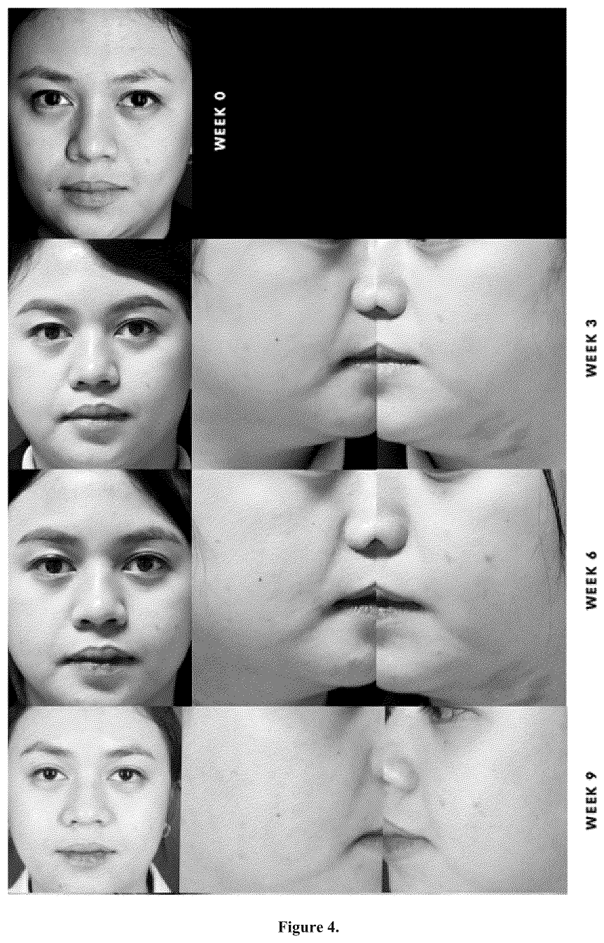

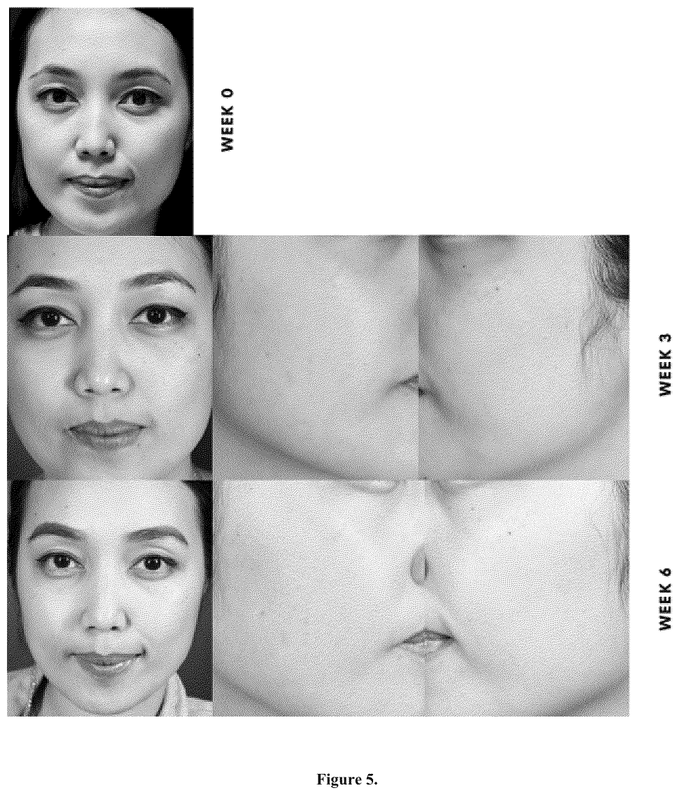

100% observed brighter skin within a week after the first session. 50% observed firmer and finer skin within one week after the first session. 100% observed smoother and brighter skin after 3 sessions. 100% observed faded acne marks after 3 sessions (See FIG. 1-FIG. 5).

No skin irritation was reported and mild skin itching was reported while the skin is healing. The results were observed early in the treatment, and improvements continued with each subsequent session. No skin irritation was reported during the study, and only mild peeling was observed as a result of the skin healing and rejuvenating following the treatment (See FIG. 1-FIG. 5).

The results of the refining composition serum and brightening composition serum are highly encouraging, demonstrating their potential to improve skin texture, tone, and brightness. After 9 weeks of treatment, all participants reported brighter and smoother skin, with less visible pores and minimized fine lines. (See FIG. 1-FIG. 5).

It is worth noting that the use of a combination of numbing cream and topical anesthesia helped to minimize discomfort during the treatment. Moreover, the use of a 2 mm Derma Roller for the first session and a 2 mm Derma Pen from the second session onwards provided the ideal depth for penetration of the serum, ensuring its maximum effectiveness.

The serum was well-tolerated, with no reports of skin irritation and only very mild peeling observed during the treatment, suggesting that it is safe for use in clinical settings.

These results suggest that the combination of ingredients in the serum, including the refining composition serum's ability to reduce fine lines and the brightening composition serum's ability to brighten the skin, may be effective in improving the appearance of various skin conditions, helping individuals achieve healthier, more vibrant-looking skin.

Based on the clinical trial of the refining composition serum and brightening composition serum, the treatment has shown promising results in improving the appearance of skin affected by various conditions.

After nine weeks of treatment, all participants reported significant improvements in skin texture, including smoother and brighter skin, even skin tone, and minimized pores and fine lines.

The treatment resulted in brighter and smoother skin with less visible pores and minimized fine lines.

The initial results were also impressive, with 100% of participants observing brighter skin within a week after the first session and 50% observing firmer and finer skin within the same period.

Furthermore, all participants reported faded acne marks after three sessions, indicating the serum's potential for treating acne scars.

The treatment was well-tolerated by all participants, with no skin irritation reported and only very mild peeling observed as a result of the skin healing and rejuvenating following the treatment.

In summary, the combination of refining composition serum and brightening composition serum holds great potential as a safe and effective approach to treating various skin imperfections.

### Example 2

A dermatologist administered 0.5 cc brightening composition as described in Table 2 to patients and 0.5 cc anti-scarring composition as described in Table 3 to patients. There are three sessions with 3 weeks interval per session.

3 Female and 2 Male Volunteers, 20-49 years old were participated in the study, which have skin type of Oily & Combination skin. Acne scars and inflammatory marks were observed before applying the composition.

Dermaroller (2 mm) was used for the first session and Dermapen (2 mm) was used from the Second session onwards. A combination of numbing cream and topical anesthesia was applied at least 45 minutes prior to each session.

The primary objective of the study was to evaluate the efficacy of the treatment in improving the appearance of acne scars, skin texture, and brightness.

The secondary objective was to assess the safety and tolerability of the treatment on the participants. This study evaluated the outcomes of three sessions with a 3-week interval per session, administered by a dermatologist.

### Method

### Study Design:

The clinical trial was a prospective, single-arm study designed to assess the efficacy of the combined use of anti-scaring composition serum and brightening composition serum in improving the appearance of acne scars and inflammatory marks in individuals with oily and combination skin.

The study involved three sessions with 3-week intervals per session. Dermaroller and Dermapen were used for the first and subsequent sessions, respectively. Numbing cream and topical anesthesia were applied before each session.

### Study Participants

The study participants were 4 female and 3 male volunteers aged between 20-49 years old. All participants had oily and combination skin and presented with acne scars and inflammatory marks.

### Intervention

The study intervention consisted of administering 0.5 cc Anti-scaring composition+0.5 cc Brightening composition serum to the participants in each session, using Dermaroller for the first session (2 mm) and Dermapen from the second session onwards (2 mm). A combination of numbing cream and topical anesthesia was applied at least 45 minutes prior to each session.

### Outcome Measures

The primary outcome measures were improvements in the appearance of acne scars, inflammatory marks, and skin texture. These outcomes were assessed by evaluating the participants' skin condition after 9 weeks of the intervention. The secondary outcome measures were the safety and tolerability of the intervention.

### Data Analysis

The data analysis was conducted by comparing the participants' skin conditions before and after the intervention using descriptive statistics. The primary outcome measures were evaluated by assessing changes in acne scars, skin texture, and brightness. The safety and tolerability of the intervention were evaluated by assessing adverse events and participant feedback.

One female volunteer experienced an allergy immediately after the 3rd session. It is uncertain sure if it was because of the product because it is the same formulation used on her 1st and 2nd session. Allergies subside after taking antihistamine.

### Result

Limitation on the results of the above subject: The subject was not able to complete the 9 weeks trial due to residence relocation.

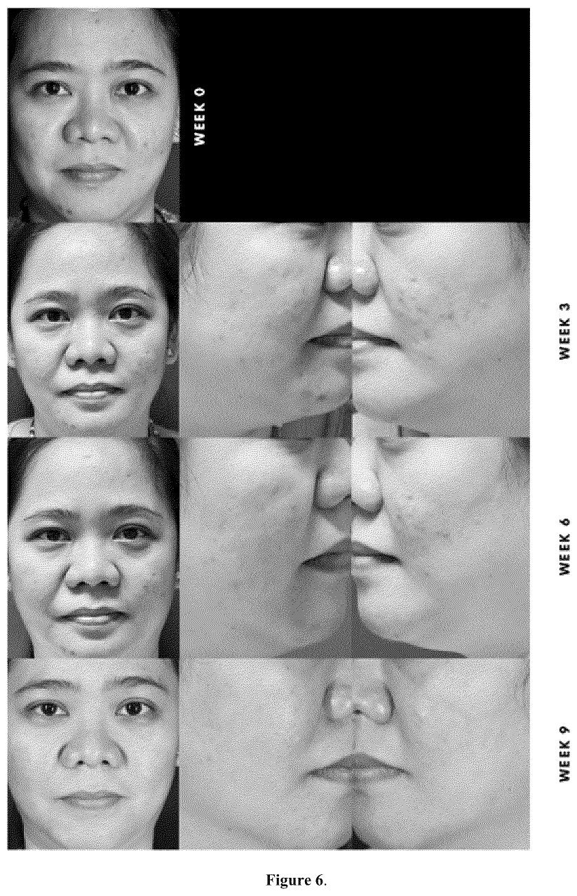

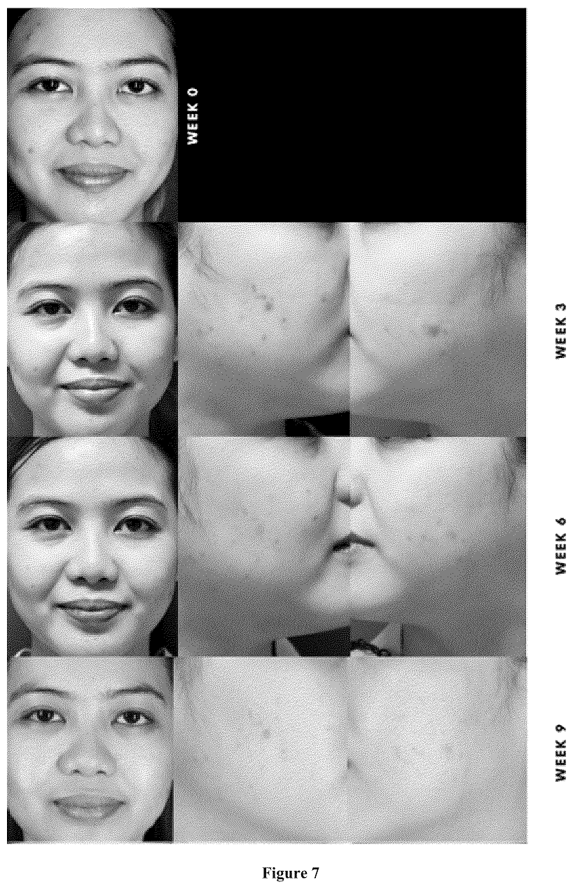

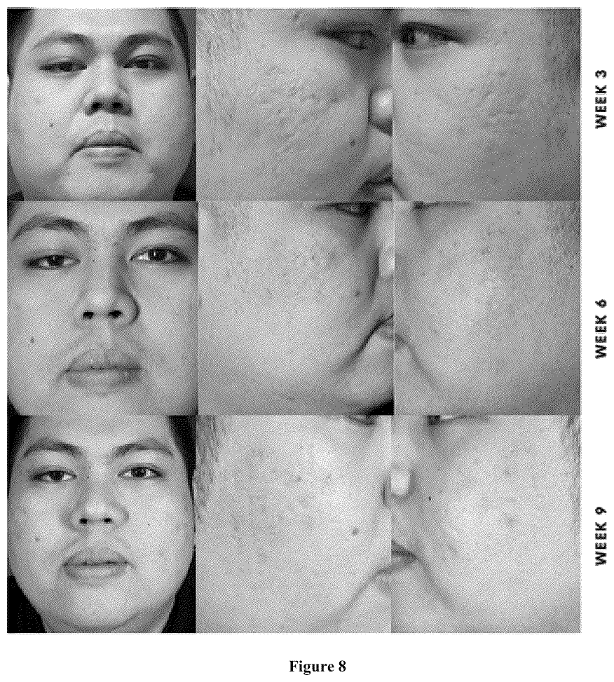

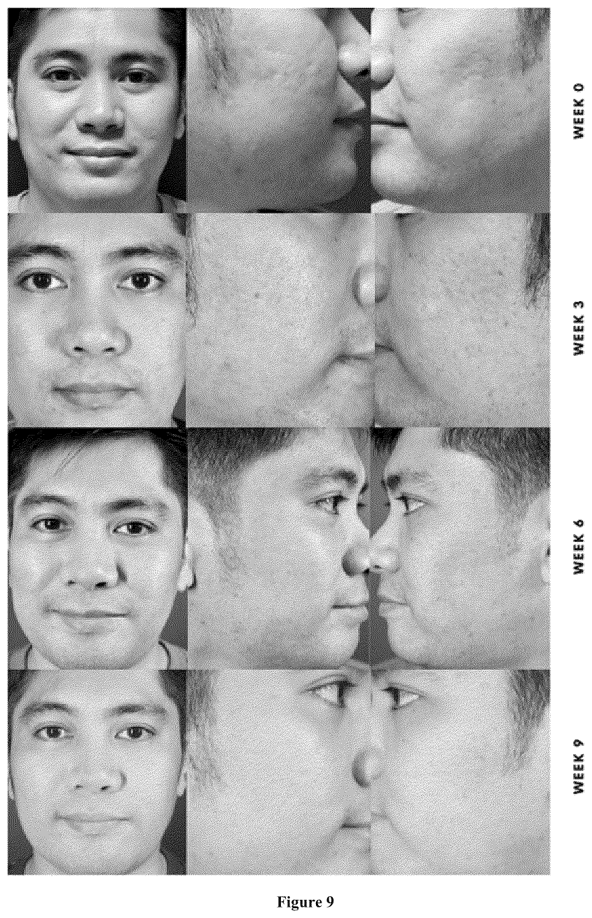

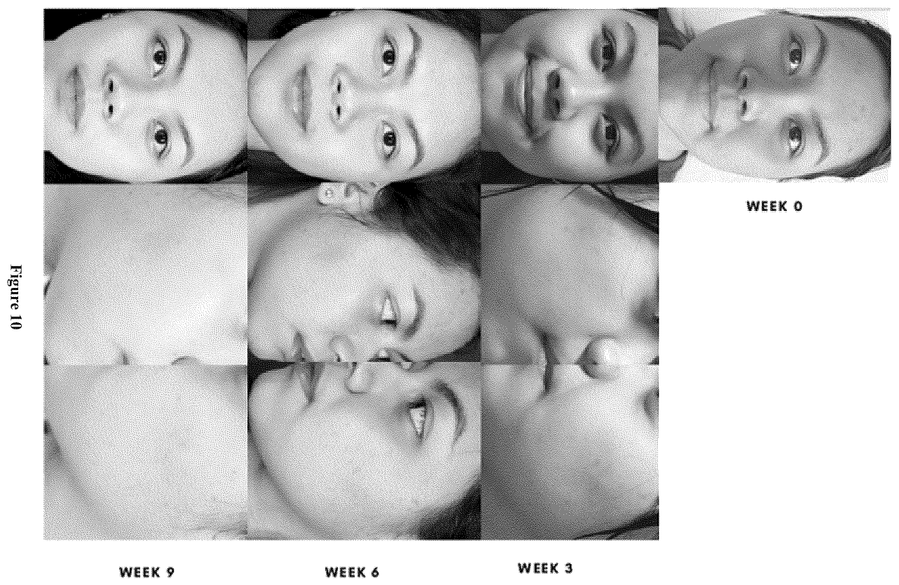

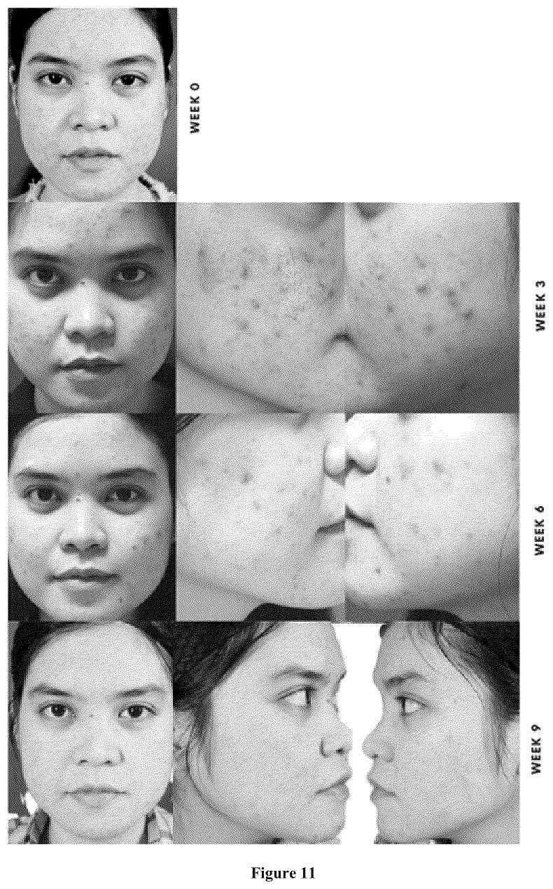

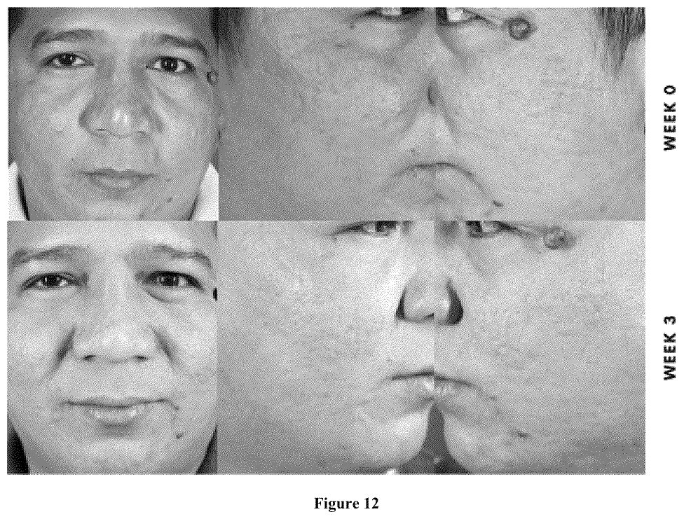

After nine weeks, all participants reported improved skin texture, with shallow acne scars, less visible pores, and brighter and tighter skin. The results observed in the participants are as follows:
100% observed firmer and finer skin within a week after the first session (See FIG. 6-FIG. 12).
100% observed shallow acne scars after the first session (See FIG. 6-FIG. 12).
100% observed brighter skin after 3 sessions (See FIG. 6-FIG. 12).
100% observed faded acne marks after 3 sessions (See FIG. 6-FIG. 12).

Micro peeling was observed (See FIG. 6-FIG. 12).

The results were observed early in the treatment, and improvements continued with each subsequent session.

No skin irritation was reported during the study, and only micro peeling was observed, which is attributed to skin healing and rejuvenating following the treatment.

NOTE: One female volunteer experienced an allergy immediately after the 3rd session. It is uncertain if it was because of the product because it is the same formulation used on her 1st and 2nd session. Allergies subside after taking antihistamine.

The results of the clinical trial of the anti-scaring composition serum and brightening composition serum indicate that the treatment is effective in reducing the appearance of acne scars, improving skin texture, and brightening the skin.

All participants reported a significant improvement in their skin's appearance after three sessions administered by a dermatologist.

It is worth noting that the use of a combination of numbing cream and topical anesthesia helped to minimize discomfort during the treatment. Moreover, the use of a 2 mm Derma Roller for the first session and a 2 mm Derma Pen from the second session onwards provided the ideal depth for penetration of the serum, ensuring its maximum effectiveness.

The serum was well-tolerated overall, with only micro peeling and mild skin itching as a result of the skin healing and rejuvenating following the treatment.

Based on the clinical trial of the anti-scaring composition serum and brightening composition serum, it can be concluded that the treatment is effective in improving the appearance of acne scars, pores, and skin texture, and in brightening the skin.

All participants reported improvement in their skin's appearance, including shallow acne scars, brighter skin, and faded acne marks, after three sessions administered by a dermatologist with a combination of numbing cream and topical anesthesia.

Furthermore, 100% of participants reported firmer and finer skin within a week after the first session, which suggests that the serum provides rapid and visible results.

However, some micro peeling and mild itching were observed as a result of the skin healing and rejuvenating following the treatment. Overall, the combination of anti-scaring composition serum and brightening composition serum shows great promise as a safe and effective approach for acne scars and other skin imperfections.

### Example 3

A dermatologist administered 0.5 cc firming composition as described in Table 4 and 0.5 cc anti-aging composition as described in Table 5 to patients. Three sessions with 3 weeks interval per session.

4 Female Volunteers, 35-65 years old were participated in the study, which have skin type of Dry & Combination skin. Hyperpigmentation/melasma were observed before applying the composition.

Dermaroller (1.5 mm) was used for the first session and Dermapen (1.5 mm-2 mm) was used from the Second session onwards. A combination of numbing cream and topical anesthesia was applied at least 45 minutes prior to each session.

The primary objective of the study was to evaluate the efficacy of the treatment in improving skin firmness, fine lines, wrinkles, and fading of melasma/hyperpigmentation. The secondary objective was to assess the safety and tolerability of the treatment on the participants. This study evaluated the outcomes of three sessions with a 3-week interval per session, administered by a dermatologist.

### Study Design

The clinical trial was a prospective, single-arm study designed to assess the efficacy of the combined use of a firming composition serum and anti-aging composition serum for improving skin firmness, fine lines, wrinkles, and fading of melasma/hyperpigmentation.

The study involved three sessions with 3-week intervals per session. Dermaroller and Dermapen were used for the first and subsequent sessions, respectively. Administering the firming composition serum and anti-aging composition serum during the procedure. Use of Numbing cream and topical anesthesia were applied before each session to reduce discomfort during the procedure.

### Study Participants

The study included 4 female volunteers aged between 35-65 years old with dry and combination skin and with hyperpigmentation/melasma and visible wrinkles.

### Intervention

The study intervention involved administering 0.5 cc firming composition+0.5 cc anti-aging composition serum during the dermneedling process in three sessions with a 3-week interval per session, using Dermaroller for the first session (1.5 mm) and Dermapen from the second session onwards (1.5 mm-2 mm). A combination of numbing cream and topical anesthesia was applied at least 45 minutes prior to procedure.

### Outcome Measures

The primary outcome measures of the study were improvements in skin firmness, fine lines, wrinkles, and fading of melasma/hyperpigmentation. These outcomes were assessed by evaluating the participants' skin condition after 9 weeks of the intervention. The secondary outcome measures were the safety and tolerability of the intervention.

### Data Analysis

The data analysis was conducted by comparing the participants' skin condition before and after the intervention using descriptive statistics. The primary outcome measures were evaluated by assessing changes in skin texture, fine lines, and skin discoloration. The safety and tolerability of the intervention were evaluated by assessing adverse events and participant feedback.

### Result

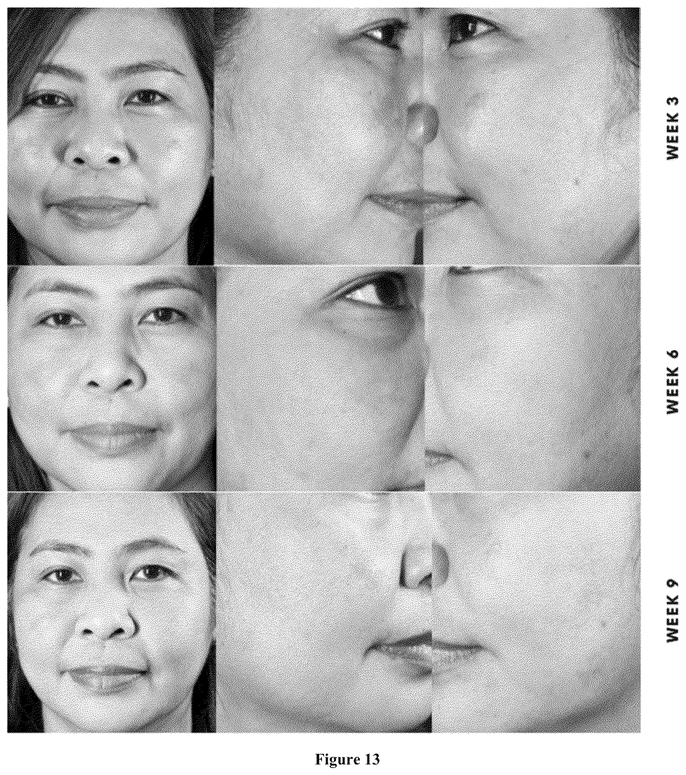

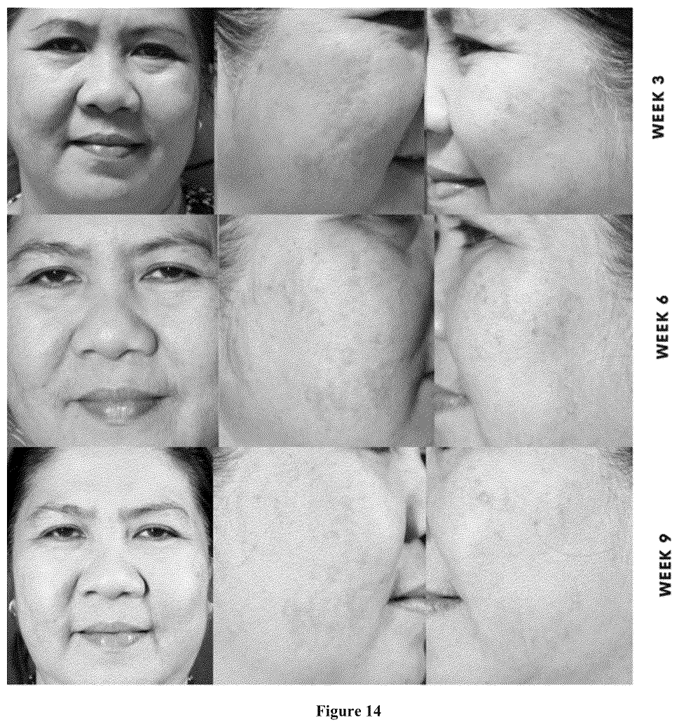

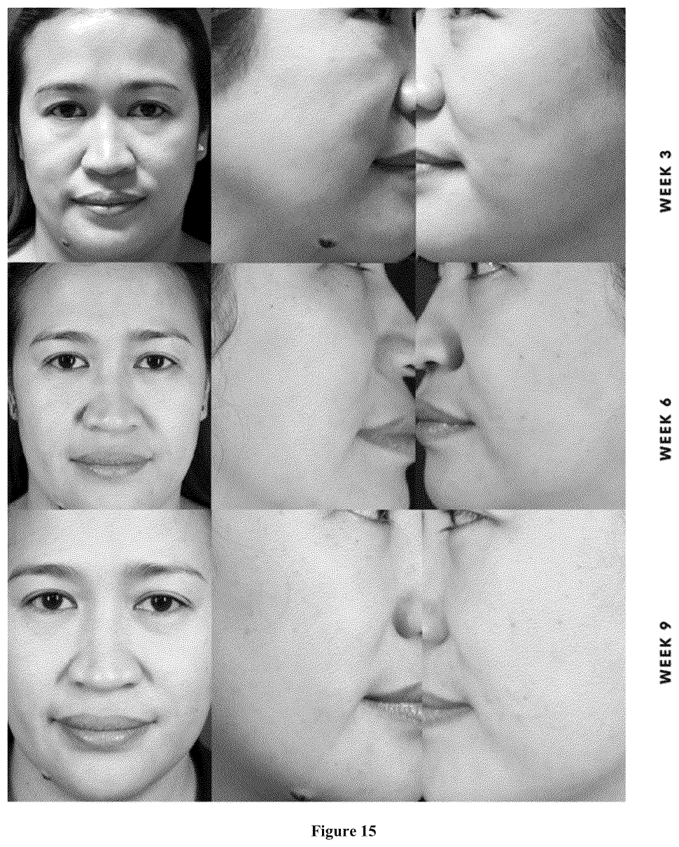

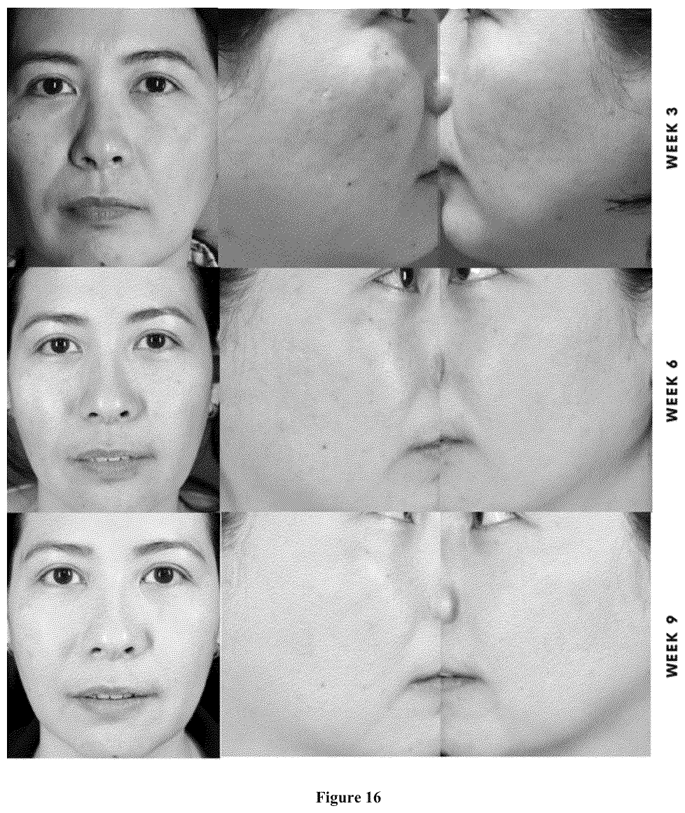

After nine weeks of treatment, all study participants reported significant improvements in their skin texture, including increased firmness, reduction in fine lines and wrinkles, and decreased hyperpigmentation and melasma. The following results were observed during the study.
100% observed firmer/finer skin within a week after first session (See FIG. 13-FIG. 16).
100% observed decrease in fine lines/wrinkles after 3 sessions (See FIG. 13-FIG. 16).
100% observed faded melasma/hyperpigmentation after 3 sessions (See FIG. 13-FIG. 16).
100% observed brighter skin after 3 sessions (See FIG. 13-FIG. 16).

No skin irritation was reported. Mild peeling was observed. Mild skin itching was reported while skin is peeling.

The results were observed early in the treatment, and improvements continued with each subsequent session.

No skin irritation was reported during the study, and only mild peeling was observed, which is attributed to the exfoliating effects of the treatment. These results indicate that the treatment is well-tolerated by patients.

The results of the nine-week study indicate that the use of the firming composition and anti-aging composition serum combination may be a promising solution for individuals concerned with sagging skin, fine lines and dark spots. The observed improvements in skin firmness, fine lines, wrinkles, and fading of Melasma/hyperpigmentation in all participants suggest that the combination of ingredients in these serums may be effective in improving the appearance of these specific skin concerns.

The treatment was well-tolerated by all participants, with no skin irritation reported during the study. Mild peeling and itching were observed during the peeling phase (2-3 days after the procedure), which is a common side effect of this type of treatment.

It is worth noting that the use of a combination of numbing cream and topical anesthesia helped to minimize discomfort during the treatment. Moreover, the use of a 1.5 mm-2 mm Derma Roller for the first session and Derma Pen from the second session onwards provided the ideal depth for penetration of the serum, ensuring its maximum effectiveness.

These results suggest that the combination of ingredients in the serum, including the firming composition serum's ability to improve skin firmness and the anti-aging composition serum's ability to fade Melasma/hyperpigmentation, may be effective in improving the appearance of various skin conditions, helping individuals achieve healthier, more youthful-looking skin.

Based on the clinical trial of the firming composition and anti-aging composition serum, the treatment has demonstrated excellent potential in improving the appearance of skin affected by sagging skin, fine lines and hyperpigmentation.

Administered by a dermatologist, the treatment involved the application of 0.5 cc firming composition+0.5 cc anti-aging composition serum for three sessions with a 3-week interval per session. The four female volunteers, aged 35-65 years old, had dry and combination skin with hyperpigmentation/melasma.

Following the sessions, all participants reported faded dark spots and tighter skin, with 100% observing firmer/finer skin within a week after the first session.

After three sessions, 100% of participants observed faded melasma/hyperpigmentation and decreased fine lines/wrinkles.

Additionally, all participants reported brighter skin after three sessions, with no skin irritation reported. Mild peeling was observed, and mild skin itching was reported while the skin was peeling.

In conclusion, the treatment combination of firming composition and anti-aging composition has shown potential as a safe and effective approach for treating sagging, fine lines and darkspot/hyper[pigmented] skin.

The treatment resulted in tighter, firmer, and brighter skin with minimized fine lines and wrinkles and no reported skin irritation.

In light of the foregoing, it should be apparent to those skilled in the art that there has been shown and described skin care compositions and their uses. However, it should also be apparent that, within the principles and scope of the invention, changes are possible. Thus, while the foregoing description and discussion address certain preferred embodiments or elements of the invention, it should further be understood that concepts of the invention, as based upon the foregoing description and discussion, may be readily incorporated into or employed in other embodiments and compositions without departing from the scope of the invention. Accordingly, the following claims are intended to protect the invention broadly as well as in the specific form shown, and all changes, modifications, variations, and other uses and applications which do not depart from the spirit and scope of the invention are deemed to be covered by the invention, which is limited only by the claims which follow.

What is claimed is:

1-268. (canceled)

269. A formulation comprising a component A and a component B, wherein the component A comprises:
a) niacinamide;
b) sodium hyaluronate;
c) 1,2-Hexanediol;
d) Sh-Oligopeptide-1;
e) Sh-Oligopeptide-2;
f) Sh-Polypeptide-1;
g) Sh-Polypeptide-9;
h) Sh-Polypeptide-11;
i) dipeptide diaminobutyroyl benzylamide diacetate; and
j) bacillus, folic acid, and/or soybean ferment extract; and
wherein the component B comprises:
a) niacinamide;
b) tranexamic acid;
c) rumex occidentalis extract;
d) ascorbic acid;
e) aminopropyl ascorbyl phosphate;
f) sxifraga sarmentosa extract;
g) arbutin;
h) paeonia suffruticosa extract;
i) scutellaria baicalensis root extract; and
j) citric acid.

270. The formulation of claim 269, wherein the component A and B, each individually, further comprises one or more of a solvent, a chelating agent, an antioxidant agent, a preservative, a skin-conditioning agent, a humectant, a thickening agent, and a dispersing agent.

271. The formulation of claim 269, wherein
a) the solvent is selected from a list consisting of water, ethanol (ethyl alcohol), isopropyl alcohol, propylene glycol, butylene glycol, glycerin, dimethyl isosorbide, caprylic/capric triglyceride, cyclopentasiloxane, dimethicone, mineral oil, silicones (e.g., cyclomethicone, dimethicone copolyol), glycols (e.g., hexylene glycol, diethylene glycol), polyethylene glycol (peg), butyl acetate, ethyl acetate, acetone, isododecane, squalane, and triethyl citrate;
b) the chelating agent is selected from a list consisting of edetate disodium (disodium EDTA), EDTA, citric acid, sodium citrate, sodium gluconate, phytic acid, gluconolactone, hydroxyethyl ethylenediamine triacetic acid (HEDTA), tetrasodium glutamate diacetate, trisodium ethylenediamine disuccinate (Trisodium EDTA), sodium phytate, potassium citrate, sodium lactate, sodium malate, sodium tartrate, and sodium ascorbate;
c) the antioxidant agent is selected from a list consisting of glutathione, tocopherol, ascorbic acid, retinol, ubiquinone, niacinamide, resveratrol, ferulic acid, alpha lipoic acid, green tea extract, grape seed extract, coenzyme Q10, astaxanthin, pomegranate extract, rosemary extract, vitamin C, vitamin E, idebenone, lycopene, beta-carotene, and tea tree oil;
d) the preservative is selected from a list consisting of phenoxyethanol, sodium sulfite, ethylhexylglycerin, parabens, phenoxyethanol, benzyl alcohol, chlorphenesin, ethylhexylglycerin, potassium sorbate, sodium benzoate, caprylyl glycol, hexylene glycol, benzoic acid, sorbic acid, sodium hydroxymethylglycinate, dehydroacetic acid, salicylic acid, ethylparaben, butylparaben, triclosan, iodopropynyl butylcarbamate (IPBC), and phthalates, formaldehyde-releasing agents;
e) the skin-conditioning agent is selected from a list consisting of acetyl glutamine, acetyl tyrosine, propylene glycol, butylene glycol, glycerin, hyaluronic acid, shea butter, cocoa butter, aloe vera, squalane, jojoba oil, almond oil, coconut oil, olive oil, panthenol, niacinamide, ceramides, collagen, elastin, peptides, allantoin, honey, vitamin E, and argan oil;
f) the humectant is selected from a list consisting of caprylyl glycol, glycerin, hyaluronic acid, sorbitol, propylene glycol, butylene glycol, urea, alpha hydroxy acids (AHAs), honey, aloe vera, sodium lactate, sodium PCA, panthenol, and allantoin;
g) the thickening agent is selected from a list consisting of hydroxyethylcellulose, carbomer, xanthan gum, cellulose gum, guar gum, acrylates/C10-30 alkyl acrylate crosspolymer, carrageenan, silica, bentonite, kaolin, starch, magnesium aluminum silicate, and hectorite; and/or
h) the dispersing agent is selected from a list consisting of lecithin, propylene glycol, glycerin, sorbitan oleate, polysorbate 20, polysorbate 80, peg-40 hydrogenated castor oil, ceteareth-20, cetearyl alcohol, steareth-20, and ethoxylated alcohols.

272. The formulation of claim 271, wherein
a) the solvent is water and/or butylene glycol;
b) the chelating agent is edetate disodium (disodium EDTA);
c) the antioxidant agent is glutathione;
d) the preservative is phenoxyethanol, sodium sulfite, and/or ethylhexylglycerin;
e) the skin-conditioning agent is acetyl glutamine, propylene glycol, and/or acetyl tyrosine;
f) the humectant is caprylyl glycol and/or glycerin;
g) the thickening agent is hydroxyethyl cellulose; and/or
h) the dispersing agent is lecithin.

273. The formulation of claim 272, wherein the component A comprises:
a) water;
b) glycerin;
c) niacinamide;
d) butylene glycol;
e) 1,2-hexanediol;
f) caprylyl glycol;
g) lecithin;
h) dipeptide diaminobutyroyl benzylamide diacetate;
i) acetyl glutamine;
j) sh-oligopeptide-1;
k) sh-oligopeptide-2;
l) sh-polypeptide-1;
m) sh-polypeptide-9,
n) sh-polypeptide-11;
o) bacillus, folic acid, and/or soybean ferment extract;
p) sodium hyaluronate;
q) glutathione;
r) hydroxyethylcellulose (HEC);
s) disodium EDTA;
t) phenoxyethanol; and
u) ethylhexylglycerin.

274. The formulation of claim 272, wherein the component B comprises:
a) water;
b) niacinamide;
c) propylene glycol;
d) glycerin;
e) butylene glycol;
f) tranexamic acid;
g) rumex occidentalis extract;
h) ascorbic acid;
i) acetyl tyrosine;
j) aminopropyl ascorbyl phosphate;
k) sxifraga sarmentosa extract;
l) arbutin;
m) paeonia suffruticosa extract;
n) scutellaria baicalensis root extract;
o) glutathione;
p) hydroxyethylcellulose (HEC);
q) citric acid;
r) sodium sulfite;
s) disodium EDTA;
t) phenoxyethanol; and
u) ethylhexylglycerin.

275. The formulation of claim 273, wherein the weight percentage of
(a) niacinamide is about 3%-6%;
(b) water, lecithin acetyl glutamine, sh-oligopeptide-1, sh-oligopeptide-2, sh-polypeptide-1, sh-polypeptide-9, sh-polypeptide-11, bacillus, folic acid, and/or soybean ferment extract, sodium hyaluronate, caprylyl glycol, butylene glycol and 1,2-hexanediol are about 2% to 5%;
(c) glycerin, water, and dipeptide diaminobutyroyl benzylamide diacetate are about 2% to 7%;
(d) sodium hyaluronate is about 0.2-0.7%;
(e) glutathione is about 0.2-0.7%;
(f) HEC is about 0.2-0.6%;
(g) glycerin is about 0.5-1.5%;
(h) disodium EDTA is about 0.05-0.15%;
(i) phenoxyethanol and ethylhexylglycerin are between 0.6-1.0%; and/or
(j) water is about 80-90%.

276. The formulation of claim 275, wherein the weight percentage of
(a) niacinamide is about 3%-5%;
(b) water, lecithin acetyl glutamine, sh-oligopeptide-1, sh-oligopeptide-2, sh-polypeptide-1, sh-polypeptide-9, sh-polypeptide-11, bacillus, folic acid, and/or soybean ferment extract, sodium hyaluronate, caprylyl glycol, butylene glycol and 1,2-hexanediol are about 2%-4%;
(c) glycerin, water, and dipeptide diaminobutyroyl benzylamide diacetate are between about 3% to 5%;
(d) sodium hyaluronate is about 0.2-0.7%; and/or
(e) glutathione is about 0.3-0.6%.

277. The formulation of claim 273, wherein the weight percentage of
a) niacinamide is about 3%-5%;
b) water, lecithin, acetyl glutamine, sh-oligopeptide-1, sh-oligopeptide-2, sh-polypeptide-1, sh-polypeptide-9, sh-polypeptide-11, bacillus, folic acid, and/or soybean ferment extract, sodium hyaluronate, caprylyl glycol, butylene glycol and 1,2-hexanediol are about 2%-4%;
c) sodium hyaluronate is about 0.3-0.6%;
d) glutathione is about 0.3-0.6%; and
e) glycerin, water, and dipeptide diaminobutyroyl benzylamide diacetate are about 3% to 5%.

278. The formulation of claim 273, wherein the weight percentage of
a) niacinamide is 4%;
b) water, lecithin acetyl glutamine, sh-Oligopeptide-1, sh-oligopeptide-2, sh-polypeptide-1, sh-polypeptide-9, sh-polypeptide-11, bacillus, folic acid, and/or soybean ferment extract, sodium hyaluronate, caprylyl glycol, butylene glycol and 1,2-hexanediol are 3%;
c) glycerin, water, and dipeptide diaminobutyroyl benzylamide diacetate are 4%;
d) sodium hyaluronate is 0.5%;
e) glutathione is 0.5%;
f) HEC is 0.4%;
g) glycerin is 1%;
h) disodium EDTA is 0.1%; and
i) phenoxyethanol and ethylhexylglycerin are 0.8%.

279. The formulation of claim 274, wherein the weight percentage of
a) water, butylene glycol, citric acid, acetyl tyrosine, sodium sulfite, aminopropyl ascorbyl phosphate, saxifraga sarmentosa extract, arbutin, paeonia suffructicosa extract, scutellaria baicalensis, root extract, and glutathione are about 2-5%;
b) niacinamide is about 2-6%;
c) water, glycerin, rumex occidentalis extract, and ascorbic acid are about 0.5-3.5%;
d) tranexamic acid is about 0.6-1.4%;
e) HEC is about 0.2-0.6%;
f) glycerin is about 0.5-1.5%;
g) disodium EDTA is about 0.05-0.15%;
h) phenoxyethanol and ethylhexylglycerin are about 0.6-1.0%;
i) propylene glycol is about 0.5-1.5%; and/or
j) water is 35-45%.

280. The composition of claim 274, wherein the weight percentage of
a) water, butylene glycol, citric acid, acetyl tyrosine, sodium sulfite, aminopropyl ascorbyl phosphate, saxifraga sarmentosa extract, arbutin, paeonia suffructicosa extract, scutellaria baicalensis, root extract, and glutathione are about 2-5%;
b) niacinamide is about 2-6%;
c) water, glycerin, rurnex occidentalis extract, and ascorbic acid are about 0.5-3.5%;
d) tranexamic acid is about 0.6-1.4%;
e) HEC is about 0.2-0.6%;
f) glycerin is about 0.5-1.5%;
g) disodium EDTA is about 0.05-0.15%;
h) phenoxyethanol and ethylhexylglycerin are about 0.6-1.0%;
i) propylene glycol is about 0.5-1.5%; and/or
j) water is 35-45%.

281. The composition of claim 280, wherein the weight percentage of
a) water, butylene glycol, citric acid, acetyl tyrosine, sodium sulfite, aminopropyl ascorbyl phosphate, saxifraga sarmentosa extract, arbutin, paeonia suffructicosa extract, scutellaria baicalensis, root extract, and glutathione are about 2-43;
b) niacinamide is about 3-5%;
c) water, glycerin, rumex occidentalis extract, and ascorbic acid is about 1-3%; and
d) tranexamic acid is about 0.8-1.2%.

282. The composition of claim 274, wherein the weight percentage of
a) water, butylene glycol, citric acid, acetyl tyrosine, sodium sulfite, aminopropyl ascorbyl phosphate, saxifraga sarmentosa extract, arbutin, paeonia suffructicosa extract, scutellaria baicalensis, root extract, and glutathione are about 3%;
b) niacinamide is about 4*;
c) water, glycerin, rumex occidentalis extract, and ascorbic acid is about 2%;
d) tranexamic acid is about 1%;
e) HEC is about 0.4%;
f) glycerin is about 1%;
g) disodium EDTA is about 0.1%;
h) phenoxyethanol and ethylhexylglycerin are about 0.8%;
i) propylene glycol is about 1%; and/or
j) water is 43.25%.

283. A method of increasing skin brightness, firmness, and/or smoothness of an individual; and/or decreasing in fine lines/wrinkle, fading melasma/hyperpigmentation, and/or fading/shallowing facial marks of an individual, wherein the method comprises applying to the individual's skin an effective amount of the formulation of claim 269.

284. The method of claim 283, wherein the effective amount comprises about 0.4-0.6 cubic centimeter of the component A and the component B individually.

285. The method of claim 284, wherein the method further comprises applying numbing cream and topical anesthesia before application of the formulation.

286. The method of claim 284, wherein the composition is applied with a dermaroller and/or a dermapen.

287. A composition of a formulation comprising a component A and a component B,
wherein the component A comprises:
a) sh-Oligopeptide-1;
b) niacinamide;
c) laminaria saccharina extract; and
d) starfish coelomic fluid extract; and
wherein the component B comprises:
a) kojic acid;
b) glycolic acid;
c) potassium azelaoyl diglycinate;
d) sodium salt of pyroglutamic acid (sodium PCA);
e) arctostaphylos uva-ursi leaf extract;
f) magnesium ascorbyl phosphate;
g) alpha-arbutin;
h) sodium benzoate; and
i) sodium metabisulfite.

288. A composition of a formulation comprising a component A and a component B,
wherein the component A comprises:
a) kojic acid;
b) glycolic acid;
c) potassium azelaoyl diglycinate;
d) sodium salt of pyroglutamic acid (sodium PCA);
e) arctostaphylos uva-ursi leaf extract;
f) magnesium ascorbyl phosphate;
g) alpha-arbutin;
h) sodium benzoate; and
i) sodium metabisulfite; and
wherein the component B comprises:
a) sh-oligopeptide-1;
b) snail secretion filtrate; and
c) copper tripeptide-1.
